(19) United States
(12) Patent Application Publication
(10) Pub. No.: US 2017/0114133 A1
(43) Pub. Date: Apr. 27, 2017

(54) TEM8 ANTIBODIES AND METHODS OF USE

(71) Applicant: BIOMED VALLEY DISCOVERIES, INC., Kansas City, MO (US)

(72) Inventors: Saurabh Saha, Wellesley Hills, MA (US); Xiaoyan M. Zhang, Lexington, MA (US)

(21) Appl. No.: 15/317,048

(22) PCT Filed: Jun. 9, 2015
(86) PCT No.: PCT/US2015/034955

§ 371 Date: Dec. 7, 2016

Related U.S. Application Data
(60) Provisional application No. 62/009,366, filed on Jun. 9, 2014.

(51) Int. Cl. C07K 16/28; C07K 14/705; A61K 38/05; G01N 33/574; C07K 16/30; C07K 14/725; A61K 31/5365

(52) U.S. Cl. CPC C07K 16/28; C07K 14/7051; C07K 14/70521; C07K 14/70578; A61K 47/48569; A61K 31/5365; G01N 33/57492; C07K 16/30; A61K 38/05; C07K 2317/77; C07K 2317/567; C07K 2317/52; C07K 2317/72; C07K 2317/524; C07K 2317/526; C07K 2317/53; C07K 2317/732; C07K 2317/41; G01N 2333/705; C07K 2319/03; C07K 2319/02; C07K 2319/74; A61K 38/00

(57) ABSTRACT

The present invention provides, inter alia, isolated monoclonal and polyclonal anti-tumor endothelial marker 8 (TEM8) antibodies or antigen binding fragments thereof that (a) bind to TEM8 membrane antigen in its native form occurring on the surface of a tumor cell; (b) may be internalized by a tumor cell; (c) bind strongly to tumor cells but not or only minimally to cells which lack expression of TEM8; and (d) are characterized in that the mean fluorescence intensity (MFI) of the antibody or an antigen binding fragment thereof against a mammalian cell line expressing TEM8 is at least two times higher than the MFI against the mammalian cell line not expressing TEM8 at antigen saturation. Chimeric antigen receptors (CARs) including an antigen binding fragment of such antibodies, modified antibodies, compositions, pharmaceutical compositions, and kits including the antibodies according to the present invention, and methods of use are also provided.

### CROSS REFERENCE TO RELATED APPLICATIONS

This application claims benefit of U.S. Patent Application Ser. No. 62/009,366, filed on Jun. 9, 2014 which application is incorporated by reference herein in its entirety.

### FIELD OF INVENTION

The present invention provides, inter alia, isolated monoclonal anti-tumor endothelial marker 8 (TEM8) antibodies (mAbs) or antigen binding fragments thereof. Methods of using such antibodies, chimeric antigen receptors (CARs) comprising an antigen binding fragment of such antibodies, modified antibodies, compositions and kits comprising such antibodies are also provided.

### INCORPORATION BY REFERENCE OF SEQUENCE LISTING

This application contains references to amino acids and/or nucleic acid sequences that have been filed concurrently herewith as sequence listing text file 0385337.txt, file size of 23.5 KB, created on Jun. 4, 2015. The aforementioned sequence listing is hereby incorporated by reference in its entirety pursuant to 37 C.F.R. §1.52(e)(5).

### BACKGROUND OF THE INVENTION

Targeting tumor-associated vasculature is considered a promising approach to cancer therapy. Various classes of chemotherapeutics directed toward tumor vasculature have been developed, including anti-angiogenic agents and vascular disrupting agents, the former affecting neovascularization and the latter targeting existing blood vessels that supply tumors with nutrients and oxygen. Though these therapies are widely used, particularly in cases of metastatic cancer, they are hampered, e.g., by their toxicity and off-target effects against healthy vasculature. Thus, there exists, inter alia, a need for additional therapeutics that more specifically target tumor-associated vasculature. The present invention is direct to meeting these and other needs.

### SUMMARY OF THE INVENTION

One embodiment of the present invention is an isolated monoclonal antibody (mAb) or an antigen binding fragment thereof. The mAb or antigen binding fragment thereof

(a) binds to tumor endothelial marker 8 (TEM8) membrane antigen in its native form occurring on the surface of a tumor cell;

(b) may be internalized by a tumor cell;

(c) binds strongly to tumor cells but not or only minimally to cells which lack expression of TEM8; and

(d) is characterized in that the mean fluorescence intensity (MFI) of the mAb or an antigen binding fragment thereof against a mammalian cell line expressing TEM8 is at least two times higher than the MFI of the mAb or an antigen binding fragment thereof against the mammalian cell line not expressing TEM8 at antigen saturation.

Another embodiment of the present invention is an isolated mAb. The isolated mAb is selected from the group consisting of those clones listed in Table 1A below and having the listed characteristics:

TABLE 1A

| | Clone | Clone | Clone | | |
|---|---|---|---|---|---|
| | binding to | binding to | binding to | | |
| | CHO-TEM8 | HEK-TEM8 | HEK | | |
| Clone | (MFI) | (MFI) | (MFI) | Heavy | Light |
| 1A2.B12 | 1445 | 365 | 161 | IgG2a | K |
| 1A2.D12 | 1166 | 750 | 69 | IgG1 | K |
| 1A2.E12 | 813 | 486 | 127 | IgG1 | K |
| 1C2.A11 | 1527 | 593 | 74 | IgG1 | K |
| 1C2.B11 | 1427 | 472 | 35 | IgG1 | K |
| 1C2.C10 | 2605 | 603 | 55 | IgG1 | K |
| 1C2.E8 | 858 | 449 | 165 | IgG1 | K |
| 3C5.A11 | 1474 | 406 | 129 | IgG2b | K |
| 3C5.B10 | 1025 | 259 | 57 | IgG2b | K |
| 6H6.C12 | 767 | 257 | 105 | IgG1 | K |
| 7B2.A11 | 1241 | 331 | 90 | IgG2a | K |
| 7B2.B10 | 1376 | 243 | 82 | IgG2b | K |
| 7B7.B12 | 1411 | 432 | 142 | IgG2b | K |
| 8D3.D11 | 172 | 223 | 43 | IgG2b | K |
| 8H2.B11 | 1055 | 150 | 50 | IgG1 | K |
| 8H2.C12 | 2237 | 405 | 74 | IgG1 | K |

A further embodiment of the present invention is a chimeric antigen receptor (CAR). The chimeric antigen receptor (CAR) comprises:

(a) an antigen binding fragment of an antibody according to the present invention, including those mAB clones identified in Table 1A; and

(b) a signaling domain of a T-cell receptor.

An additional embodiment of the present invention is a modified antibody that binds a TEM8 antigen. The modified antibody comprises a variant human IgG1 Fc region, wherein the variant human IgG1 Fc region comprises at least one amino acid modification relative to the human IgG1 Fc region of a parent antibody according to Table 1A that binds the TEM8 antigen, the amino acid modification(s) comprising amino acid modification(s) that alter the affinity or avidity of the variant Fc region for binding to an FcγR, such that the modified antibody exhibits, in an in vitro assay, enhanced effector function activity mediated by the FcγR binding in cells positive for the TEM8 antigen, and the parent antibody exhibits lower or non-detectable effector function activity in the cells using the in vitro assay, the amino acid modification(s) that alter the affinity or avidity of the variant Fc region for binding to an FcγR consisting of the modification of 1, 2, 3, 4 or 5 amino acid residues of the IgG1 Fc region of the parent antibody.

Another embodiment of the present invention is a modified antibody that binds a TEM8 antigen. The modified antibody comprises a variant human IgG1 Fc region, wherein the variant human IgG1 Fc region comprises at least one amino acid modification relative to the human IgG1 Fc region of a parent antibody identified in Table 1A that binds the TEM8 antigen, the amino acid modification(s) comprising amino acid modification(s) that alter the affinity or avidity of the variant Fc region for binding to an FcγR, such that the modified antibody exhibits enhanced effector function activity mediated by the FcγR binding in cells positive for the antigen and the parent antibody exhibits lower or non-detectable effector function activity; such that the modified antibody is therapeutically effective in a subject refractory to treatment with the parent antibody, the amino acid modification(s) that alter the affinity or avidity of the variant Fc region for binding to an FcγR consisting of the modification of 1, 2, 3, 4 or 5 amino acid residues of the IgG1 Fc region of the parent antibody.

A further embodiment of the present invention is a composition. The composition comprises an effector agent or a detectable marker, which agent or marker is conjugated to a mAb or antigen binding fragment thereof, wherein the mAb or antigen binding fragment thereof:

(a) binds to tumor endothelial marker 8 membrane (TEM8) antigen in its native form occurring on the surface of a tumor cell;

(b) may be internalized by a tumor cell;

(c) binds strongly to tumor cells but not or only minimally to cells which lack expression of TEM8; and

(d) is characterized in that the mean fluorescence intensity (MFI) of the mAb or an antigen binding fragment thereof against a mammalian cell line expressing TEM8 is at least two times higher than the MFI of the mAb or an antigen binding fragment thereof against the mammalian cell line not expressing TEM8 at antigen saturation.

An additional embodiment of the present invention is a pharmaceutical composition. The pharmaceutical composition comprises an effective amount of any mAb, antigen binding fragment thereof, CAR, modified antibody or composition of the present invention and a pharmaceutically acceptable carrier.

A further embodiment of the present invention is a kit for treating or ameliorating the effects of a cancer in a subject. The kit comprises any mAb, an antigen binding fragment thereof, CAR, modified antibody, composition or pharmaceutical composition of the present invention packaged in combination with instructions for its use.

Another embodiment of the present invention is a kit for the detection of a tumor cell. The kit comprises any isolated mAb or an antigen binding fragment thereof of the present invention packaged in combination with instructions for its use.

A further embodiment of the present invention is a kit for the detection of pathological angiogenesis in a subject. The kit comprises any isolated mAb or an antigen binding fragment thereof of the present invention packaged in combination with instructions for its use.

An additional embodiment of the present invention is a method for identifying tumor cells. The method comprises:

(a) contacting a cell to be identified with any isolated mAb or an antigen binding fragment thereof of the present invention; and

(b) identifying those cells to which the mAb or antigen binding fragment thereof specifically binds, wherein those cells bound to the mAb or antigen binding fragment thereof are tumor cells.

Another embodiment of the present invention is a method for treating or ameliorating the effects of a disease in a subject. The method comprises administering to a subject in need thereof an effective amount of any isolated mAb or an antigen binding fragment thereof, CAR, modified antibody, composition, or pharmaceutical composition of the present invention.

An additional embodiment of the present invention is a method of modulating the binding of an anthrax protective antigen to a cell. The method comprises: contacting the cell with an effective amount of any isolated mAb or an antigen binding fragment thereof disclosed herein to modulate the binding of the anthrax protective antigen to the cell.

An additional embodiment of the present invention is a polyclonal antibody which:

(a) binds to tumor endothelial marker 8 (TEM8) membrane antigen in its native form occurring on the surface of a tumor cell;

(b) may be internalized by a tumor cell;

(c) binds strongly to tumor cells but not or only minimally to cells which lack expression of TEM8; and

(d) is characterized in that the mean fluorescence intensity (MFI) of the polyclonal antibody against a mammalian cell line expressing TEM8 is at least two times higher than the MFI of the polyclonal antibody against the mammalian cell line not expressing TEM8 at antigen saturation.

A further embodiment of the present invention is a polyclonal antibody selected from the group consisting of those clones listed in Table 1B below and having the listed characteristics:

TABLE 1B

| | binding to | binding to | binding to | | |
|---|---|---|---|---|---|
| | CHO-TEM8 | HEK-TEM8 | HEK | | |
| Name | (MFI) | (MFI) | (MFI) | Heavy | Light |
| 3C5.E7 | 536 | 215 | 92 | IgG2b/IgG1 | K |
| 6H6.B11 | 963 | 232 | 96 | IgG1 | K |
| 7B2.D9 | 678 | 284 | 102 | IgG2a | K |
| 7B7.E9 | 852 | 324 | 96 | IgG2a | K/λ |

Another embodiment of the present invention is a composition comprising an effector agent or a detectable marker, which agent or marker is conjugated to a polyclonal antibody, wherein the polyclonal antibody:

(a) binds to tumor endothelial marker 8 (TEM8) membrane antigen in its native form occurring on the surface of a tumor cell;

(b) may be internalized by a tumor cell;

(c) binds strongly to tumor cells but not or only minimally to cells which lack expression of TEM8; and

(d) is characterized in that the mean fluorescence intensity (MFI) of the polyclonal antibody against a mammalian cell line expressing TEM8 is at least two times higher than the MFI of the polyclonal antibody against the mammalian cell line not expressing TEM8 at antigen saturation.

An additional embodiment of the present invention is a pharmaceutical composition. This pharmaceutical composition comprises an effective amount of any polyclonal antibody according to the present invention and a pharmaceutically acceptable carrier.

A further embodiment of the present invention is a kit for treating or ameliorating the effects of a cancer in a subject. This kit comprises any polyclonal antibody or pharmaceutical composition according to the present invention packaged in combination with instructions for use.

Another embodiment of the present invention is a kit for the detection of a tumor cell. This kit comprises any polyclonal antibody according to the present invention packaged in combination with instructions for use.

An additional embodiment of the present invention is a method for treating or ameliorating the effects of a disease in a subject. This method comprises administering to a subject in need thereof an effective amount of any polyclonal antibody or pharmaceutical composition according to the present invention.

### BRIEF DESCRIPTION OF THE DRAWINGS

FIG. 1 shows the plasmid map of GS50782 pcDNA3.1(+)-ANTXR1.

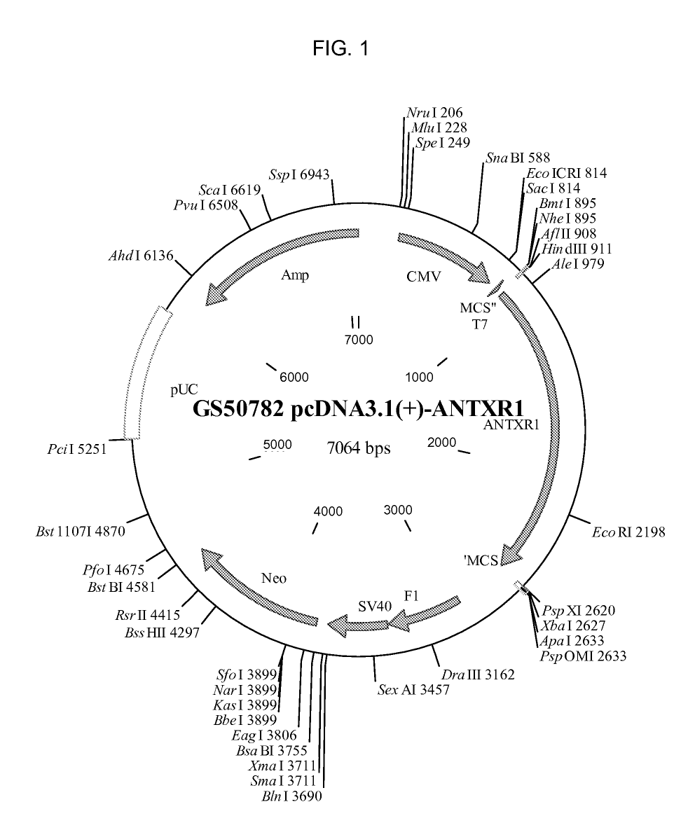

FIG. 2 shows the plasmid map of GS50831 pcDNA3.1(+)-CMG2.

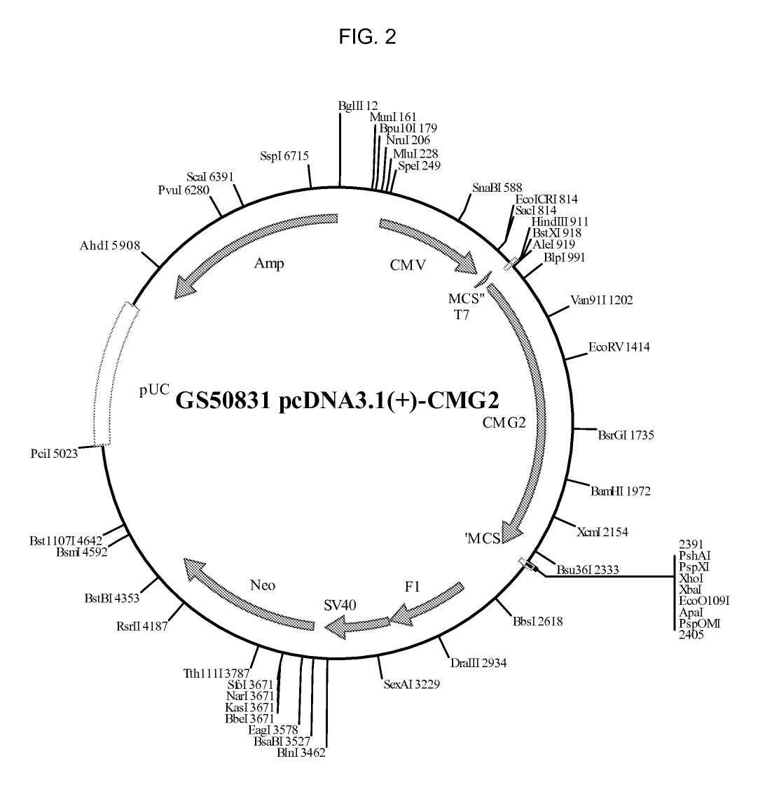

### DETAILED DESCRIPTION OF THE INVENTION

Solid tumor growth is typically accompanied by neovascularization in and around a nascent tumor in order to supply the malignancy with oxygen and other nutrients. As has been shown by St. Croix and colleagues (St. Croix, et al., 2000), tumor vasculature is distinct from normal vasculature in that several genes are differentially expressed in tumor-associated blood vessels. One of these genes, tumor endothelial marker 8 (TEM8) membrane antigen, is upregulated in the vasculature of malignant solid tumors, with limited expression in healthy tissues. Representative TEM8 nucleic acid and polypeptide sequences are shown in Table 2, below.

TABLE 2
TEM8 Sequences

| SEQ ID | Sequence | Nucleic | | Other |
|---|---|---|---|---|
| NO. | Name | Acid/Polypeptide | Organism | Information |
| 1 | TEM8 mRNA | Nucleic acid | Homo | Transcript |
| | | | sapiens | variant 1 |
| 2 | TEM8 | Polypeptide | Homo | Isoform 1 |
| | protein | | sapiens | precursor |
| 3 | TEM8 mRNA | Nucleic acid | Homo | Transcript |
| | | | sapiens | variant 2 |
| 4 | TEM8 | Polypeptide | Homo | Isoform 2 |
| | protein | | sapiens | precursor |
| 5 | TEM8 mRNA | Nucleic acid | Homo | Transcript |
| | | | sapiens | variant 3 |
| 6 | TEM8 | Polypeptide | Homo | Isoform 3 |
| | protein | | sapiens | precursor |

Therapies targeting the immune system to cells expressing TEM8 may be an effective means of selectively killing tumor cells, particularly those centrally located within a tumor that may not receive optimal exposure to routine chemotherapeutic agents.

As such, one embodiment of the present invention is an isolated monoclonal antibody (mAb) or an antigen binding fragment thereof. The isolated monoclonal antibody or antigen binding fragment thereof

(a) binds to tumor endothelial marker 8 (TEM8) membrane antigen in its native form occurring on the surface of a tumor cell;

(b) may be internalized by a tumor cell;

(c) binds strongly to tumor cells but not or only minimally to cells which lack expression of TEM8; and

(d) is characterized in that the mean fluorescence intensity (MFI) of the mAb or an antigen binding fragment thereof against a mammalian cell line expressing TEM8 is at least two times higher than the MFI of the mAb or an antigen binding fragment thereof against the mammalian cell line not expressing TEM8 at antigen saturation.

As used herein, an “antibody” and “antigen-binding fragments thereof” encompasses naturally occurring immunoglobulins (e.g., IgM, IgG, IgD, IgA, IgE, etc.) as well as non-naturally occurring immunoglobulins, including, for example, single chain antibodies, chimeric antibodies (e.g., humanized murine antibodies) heteroconjugate antibodies (e.g., bispecific antibodies), Fab′, F(ab′)₂, Fab, Fv, and rIgG. See also, e.g., Pierce Catalog and Handbook, 1994-1995 (Pierce Chemical Co., Rockford, Ill.); Kuby, et al., 1998. As used herein, “antigen-binding fragments” mean that a portion of the full length antibody that retains the ability to recognize the antigen, as well as various combinations of such portions.

Naturally occurring immunoglobulins, such as IgG, are well known to those of skill in the art. IgGs are the primary antibodies found in circulation and are responsible for the majority of antibody-mediated immune functions. There are four human IgG subclasses (IgG1, 2, 3, and 4), named in order of their abundance in serum (IgG1 being the most abundant). IgGs have two identical heavy chains and two identical light chains. Variations in heavy chain type define the numerous antibody isotypes, with IgA having alpha heavy chains and IgG having gamma heavy chains, for example.

Non-naturally occurring antibodies can be constructed using solid phase peptide synthesis, can be produced recombinantly, or can be obtained, for example, by screening combinatorial libraries consisting of variable heavy chains and variable light chains as described by Huse et aL, Science 246:1275-1281 (1989), which is incorporated herein by reference. These and other methods of making, for example, chimeric, humanized, CDR-grafted, single chain, and bifunctional antibodies, are well known to those skilled in the art (Winter and Harris, Immunol. Today 14:243-246 (1993); Ward et al., Nature 341:544-546 (1989); Harlow and Lane, supra, 1988; Hilyard et al., Protein Engineering: A practical approach (IRL Press 1992); Borrabeck, Antibody Engineering, 2d ed. (Oxford University Press 1995); each of which is incorporated herein by reference).

Full length antibodies can be proteolytically digested down to several discrete, functional antibody fragments, which retain the ability to recognize the antigen. For example, the enzyme papain can be used to cleave a full length immunoglobulin into two Fab fragments and an Fc fragment. Thus, the Fab fragment is typically composed of two variable domains and two constant domains from the heavy and light chains. The Fv region is usually recognized as a component of the Fab region and typically comprises two variable domains, one from each of the heavy (V_{H}, “heavy chain variable region”, as used herein) and light (V_{L} “light chain variable region”, as used herein) chains. The enzyme pepsin cleaves below the hinge region, so a F(ab′)₂ fragment and a pFc′ fragment is formed. F(ab′)₂ fragments are intact antibodies that have been digested, removing the constant (Fc) region. Two Fab′ fragments can then result from further digestion of F(ab′)₂ fragments. As used herein, “antibody fragments” means that a portion of the full length antibody that retains the ability to recognize the antigen, as well as various combinations of such portions. Examples of antigen-binding fragments include, but are not limited to, Fv, Fab, Fab′, Fab′-SH, F(ab′)₂, diabodies, tribodies, scFvs, and single-domain antibodies (dAbs).

Typically, a full length antibody has at least one heavy and at least one light chain. Each heavy chain contains a variable domain (V_{H}) and typically three or more constant domains (C_{H}1, C_{H}2, C_{H}3, etc.), while each light chain contains a variable domain (V_{L}) and a constant domain C_{L}. Light and heavy chain variable regions contain four “framework” regions interrupted by three hypervariable regions, also called “complementarity-determining regions” or “CDRs”. The extent of the framework regions and CDRs have been defined. See, e.g., Kabat et al., U.S. Dept. of Health and Human Services, Sequences of Proteins of Immunological Interest (1983) and Chothia et al., J. Mol. Biol. 196:901-917 (1987). The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody, whereas a V_{L} CDR1 is the CDR1 from the variable domain of the light chain of the antibody.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256: 495 (1975), and as modified by the somatic hybridization method as set forth above; or may be made by other recombinant DNA methods (see, e.g., U.S. Pat. No. 4,816,567). The term “polyclonal antibody” denotes herein a substantially heterogeneous population of antibodies which react with more than one epitope of an antigen, and also is not to be construed as requiring production of the antibody by any particular method. Polyclonal antibodies encompass those isolated or purified from mammalian (including transgenic animal) blood, secretions, or other fluids, or from eggs, (see, e.g., U.S. Pat. No. 5,939,598), as well as a mixture of different monoclonal antibodies, and finally a polyclonal antibody may be produced as a recombinant polyclonal antibody (see, e.g., U.S. Pat. No. 5,789,208).

In the present invention, “antibodies” or “antibody” means both monoclonal and polyclonal antibodies of the present invention, unless the context makes clear that a particular type of antibody is intended. Additional types of antibodies that may be part of the monoclonal or polyclonal antibodies of the present invention include, but are not limited to, chimeric, humanized, and human antibodies. For application in humans, it is often desirable to reduce immunogenicity of antibodies originally derived from other species, like mouse. This can be done by construction of chimeric antibodies, or by a process called “humanization”. In this context, a “chimeric antibody” is understood to be an antibody comprising a domain (e.g. a variable domain) derived from one species (e.g. mouse) fused to a domain (e.g. the constant domains) derived from a different species (e.g. human).

As used herein, the term “humanized antibody” refers to forms of antibodies that contain sequences from non-human (e.g., murine) antibodies as well as human antibodies. Such antibodies are chimeric antibodies which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the framework (FR) regions are those of a human immunoglobulin sequence (“human framework region”, as used herein). The humanized antibody optionally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol 2:593-596 (1992)). Humanization can be essentially performed following the method of Winter and co-workers (Jones et al., Nature 321:522-525 (1986); Riechmann et al., Nature 332:323-3′27 (1988); Verhoeyen et al., Science 239:1534-1536 (1988)), by substituting rodent CDRs or CDR sequences for the corresponding sequences of a human antibody.

Furthermore, technologies have been developed for creating antibodies based on sequences derived from the human genome, for example by phage display or using transgenic animals (WO 90/05144; D. Marks, H. R. Hoogenboom, T. P. Bonnert, J. McCafferty, A. D. Griffiths and G. Winter (1991) “By-passing immunisation. Human antibodies from V-gene libraries displayed on phage.” J. Mol. Biol., 222, 581-597; Knappik et al., J. Mol. Biol. 296: 57-86, 2000; S. Carmen and L. Jermutus, “Concepts in antibody phage display”. Briefings in Functional Genomics and Proteomics 2002 1(2):189-203; Lonberg N, Huszar D. “Human antibodies from transgenic mice”. Int Rev Immunol. 1995; 13(1):65-93; Bruggemann M, Taussig M J. “Production of human antibody repertoires in transgenic mice”. Curr. Opin. Biotechnol. 1997 August; 8(4):455-8.). Such antibodies are “human antibodies” in the context of the present invention.

As used herein, “recombinant” antibody means any antibody whose production involves expression of a non-native DNA sequence encoding the desired antibody structure in an organism. In the present invention, recombinant antibodies include, e.g., tandem scFv (taFv or scFv₂), diabody, dAb₂/VHH₂, knob-into-holes derivatives, SEED-IgG, heteroFc-scFv, Fab-scFv, scFv-Jun/Fos, Fab′-Jun/Fos, tribody, DNL-F(ab)₃, scFv₃-CH1/CL, Fab-scFv₂, IgG-scFab, IgG-scFv, scFv-IgG, scFv₂-Fc, F(ab′)₂-scFv₂, scDB-Fc, scDb-CH3, Db-Fc, scFv₂-H/L, DVD-Ig, tandAb, scFv-dhlx-scFv, dAb₂-IgG, dAb-IgG, dAb-Fc-dAb, and combinations thereof.

Variable regions of antibodies are typically isolated as single-chain Fv (scFv) or Fab fragments. ScFv fragments are composed of V_{H} and V_{L} domains linked by a short 10-25 amino acid linker. Once isolated, scFv fragments can be genetically linked with a flexible peptide linker such as, for example, one or more repeats of Ala-Ala-Ala, Gly-Gly-Gly-Gly-Ser, etc. The resultant peptide, a tandem scFv (taFv or scFv₂) can be arranged in various ways, with V_{H}-V_{L} or V_{L}-V_{H} ordering for each scFv of the taFv. (Kontermann, R. E. In: Bispecific Antibodies. Kontermann R E (ed.), Springer Heidelberg Dordrecht London New York, pp. 1-28 (2011)).

As used herein, the term “epitope” refers to the portion of the antigen which is recognized by the antibody or antigen binding fragment. A single antigen (such as an antigenic polypeptide) may have more than one epitope. Epitopes may be defined as structural or functional. Functional epitopes are generally a subset of the structural epitopes and have those residues that directly contribute to the affinity of the interaction. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes formed from contiguous amino acids are typically retained on exposure to denaturing solvents, whereas epitopes formed by tertiary folding are typically lost on treatment with denaturing solvents.

As used herein, the phrase “native form” refers to any conformation of TEM8 that is present on a cell surface in a normal cellular environment. TEM8 shows significant structural similarities to integrins, which exist in two conformations, “open” and “closed”, on a cell surface. According to St. Croix and colleagues, the predominant form of TEM8 cannot be bound by SB5 antibodies, whereas AF334 antibodies can bind both forms (St. Croix, et al., 2011).

As used herein, “tumor cells” refer to any cell that is a part of abnormal growth of tissues or is associated with such growth, and include tumor epithelial cells, tumor endothelial cells, and tumor stroma, such as fibroblasts and pericytes.

As used herein, the phrase “surface of a tumor cell” refers to the exterior portion of a tumor cell that is in direct contact with the tumor cell's extracellular surroundings. Substances found natively on a cell surface or that are bound to a species native to a cell surface may be brought into the cell interior (“internalized”, as used herein) by a process known as endocytosis, which is well known to a person of skill in the art. Internalized substances may be, for example, degraded, and if they are bound to another substance, i.e. a chemotherapeutic agent, the chemotherapeutic may be released into the cell upon degradation of its binding partner.

The phrases “binds strongly” or “strong binding”, as used herein, have the same meaning as “specifically binds,” “specific binding” and the like and refer to a binding reaction between two molecules that is at least two times the background and more typically more than 10 to 100 times background molecular associations under physiological conditions. When using one or more detectable binding agents that are proteins, specific binding is determinative of the presence of the protein, in a heterogeneous population of proteins and other biologics. Thus, under designated immunoassay conditions, the specified antibodies bind to a particular protein sequence, thereby identifying its presence.

As used herein, “minimal” binding means a binding reaction between two molecules that is the same as the background under physiological conditions.

Methods of determining strong binding may be accomplished using assays that measure binding affinity and specificity, which methods are well known in the art (see, for example, Harlow and Lane, Antibodies: A laboratory manual (Cold Spring Harbor Laboratory Press, 1988); Friefelder, “Physical Biochemistry: Applications to biochemistry and molecular biology” (W.H. Freeman and Co. 1976)). One preferred method for determining strong binding is disclosed in more detail in Example 1. Typically, a readout for such an assay will be mean fluorescent intensity (MFI). As used herein, “mean fluorescence intensity (MFI)” is the average fluorescence measurement detected by fluorescence activated cell sorting (FACS).

In the present invention, an antibody may be characterized by having specific binding activity (Kₐ) for an antigen of at least about 10⁵ mol⁻¹, 10⁶ mol⁻¹ or greater, preferably 10⁷ mol⁻¹ or greater, more preferably 10⁸ mol⁻¹ or greater, and most preferably 10⁹ mol⁻¹ or greater. The binding affinity of an antibody can be readily determined by one of ordinary skill in the art, for example, by Scatchard analysis (Scatchard, Ann. NY Acad. Sci. 51: 660-72, 1949).

As used herein, “cells which lack expression of TEM8” are cells that produce no TEM8 mRNA transcripts or protein products or cells that produce TEM8 transcripts and proteins at levels that are essentially undetectable using standard laboratory procedures or that are only minimally detectable by such procedures. In most cases, cells which lack expression of TEM8 will exhibit lower levels of TEM8 protein when compared to tumor cells.

As used herein, the term “antigen saturation” refers to the status of a cell wherein all binding sites, for example, TEM8 membrane antigens, for a binding agent, e.g. a TEM8 antibody, are filled and bound to said binding agent. At antigen saturation, higher doses of binding agent may not result in greater downstream effects. Antigen saturation may be achieved by, for example, large doses of an antigen binding agent, short internalization times, or a combination of the two.

The mammalian cell line is preferably a human, a hamster, or a mouse cell line, such as Chinese hamster ovary (CHO) (including all of its progeny and variants, such as K1-, DukX B11-, DG44-, and variant Lec13 cell lines), human embryonic kidney (HEK), human embryonic retinal cell line Per.C6 (Crucell, Leiden, Netherlands), mouse myeloma NSO, baby hamster kidney (BHK), and human neuronal precursor cell line AGE1.HN (Probiogen, Berlin, Germany). Particularly preferred cell lines include CHO and HEK cell lines.

In one aspect of this embodiment, and the MFI of the mAb or an antigen binding fragment thereof against the CHO cell line expressing TEM8 is at least three times, such as at least four, five, six, seven eight, nine or ten times higher than the MFI of the mAb or an antigen binding fragment thereof against a CHO cell line not expressing TEM8 at antigen saturation.

In another aspect of this embodiment, the isolated mAb or an antigen binding fragment thereof is selected from the group consisting of 1A2.B12, 1A2.D12, 1A2.E12, 1C2.A11, 1C2.B11, 1C2.C10, 1C2.E8, 3C5.A11, 3C5.B10, 6H6.C12, 7B2.A11, 7B2.B10, 7B7.B12, 8D3.D11, 8H2.B11, 8H2.C12, and antigen binding fragments thereof.

In a further aspect of this embodiment, the isolated mAb or an antigen binding fragment thereof further comprises a human framework region. In the present invention “a human framework region” means all or substantially all of the framework (FR) regions (or regions in the variable heavy chain or the variable light chain that are not complementarity-determining regions) are those of a human immunoglobulin sequence. Human framework region may be obtained from known human antibody sequences, such as those available on public databases.

In an additional aspect of this embodiment, the isolated mAb or an antigen binding fragment thereof is a humanized antibody, a chimeric antibody, or a recombinant antibody.

In another aspect of this embodiment, the antibody is preferably an IgG, although other Ig subtypes may be used.

In a further aspect of this embodiment, the antigen binding fragment of the mAb is a Fv, a Fab, a F(ab′)2, a scFV or a scFV2 fragment.

In an additional aspect of this embodiment, the isolated mAb or an antigen binding fragment thereof is conjugated to a label or to an effector agent. As used herein, the term “conjugated” and grammatical variations thereof refer to an isolated mAb or an antigen binding fragment thereof of the present invention bound to, associated with, or otherwise connected to a label or an effector agent of the present invention. Conjugation is not meant to be limited to direct connections between isolated mAbs or antigen binding fragments thereof and labels or effector agents. These species may still be considered conjugated if connected by a linker.

As used herein, a “label” is any substance that, when paired with an isolated mAb or antigen binding fragment thereof of the present invention has intrinsic properties that can indicate its presence, and therefore the presence of an isolated mAb or antigen binding fragment thereof of the present invention, in a given system. Preferably, the label is selected from the group consisting of a fluorescent marker, an enzymatic marker, a heavy metal, a radioactive marker, and combinations thereof.

Fluorescent markers are well known to those of skill in the art. Briefly, a fluorescent marker of the present invention is any substance that emits light of, usually a higher wavelength, after absorbing light of a lower wavelength. Fluorescent markers may be, but are not limited to, dyes and proteins. For example, fluorescent dyes include, but are not limited to, hydroxycoumarin, aminocoumarin, methoxycoumarin, cascade blue, pacific blue, pacific orange, lucifer yellow, NBD, R-phycoerythrin (PE), PE-Cy5 conjugates, PE-Cy7 conjugates, Red 613, PerCP, TruRed, FluorX, fluorescein, BODIPY-FL, Cy2, Cy3, Cy3B, Cy3.5, Cy5, Cy5.5, Cy7, SeTau-647, TRITC, X-rhodamine, lissamine rhodamine B, texas red, allophycocyanin (APC), and APC-Cy7 conjugates. Fluorescent proteins include, but are not limited to, fluorescent proteins derived from Aequorea Victoria, such as green fluorescent protein (GFP), EGFP, Emerald, Superfolder GFP, Azami Green, mWasabi, TagGFP, TurboGFP, AcGFP, ZsGreen, T-Sapphire, EBFP, EBFP2, Azurite, mTagBFP, ECFP, mECFP, Cerulean, mTurquoise, CyPet, AmCyan1, Midori-Ishi Cyan, TagCFP, mTFP1 (Teal), EYFP, Topaz, Venus, mCitrine, YPet, TagYFP, PhiYFP, ZsYellow1, mBanana, Kusabira Orange, Kusabira Orange2, mOrange, mOrange2, dTomato, dTomato-Tandem, TagRFP, TagRFP-T, DsRed, DsRed2, DsRed-Express (T1), DsRed-Monomer, mTangerine, mRuby, mApple, mStrawberry, AsRed2, mRFP1, JRed, mCherry, HcRed1, mRaspberry, dKeima-Tandem, HcRed-Tandem, mPlum, and AQ143, as well as fluorescent proteins from Renilla, Anemonia majano, and Branchiostoma floridae.

Enzymatic markers are well known to those of skill in the art. Briefly, an enzymatic marker of the present invention may be an enzyme or an enzyme substrate that, when exposed to its respective enzyme substrate or enzyme, produces a measurable effect. For example, a measurable effect may be a visual change, such as a change in color. Non-limiting examples of enzymatic markers include horseradish peroxidase (HRPO), urease, alkaline phosphatase, glucose oxidase, and β-galactosidase.

Heavy metals are well known to those of skill in the art. In the context of the present invention, heavy metals may be useful as contrast agents in certain imaging applications, such as X-ray imaging or electron microscopy. Non-limiting examples of heavy metals for conjugation to antibodies include colloidal metals such as gold, silver, palladium, gatalinium, tungsten, rhenium, molybdenum, bismuth, and osmium.

Radioactive markers are well known to those of skill in the art. In the context of the present invention, radioactive markers may be useful in certain imaging applications such as positron emission tomography. Non-limiting examples of radioactive markers include I-125, At-211, Lu-177, Cu-67, I-131, Sm-153, Re-186, P-32, Re-188, In-114m, and Y-90.

As used herein, an “effector agent” is any substance that can modulate cellular activity. Because the effector agent is conjugated to the antibody, the antibody can deliver the effector agent to a specific site, where the effector agents may act. Preferably, the effector agent is selected from the group consisting of a chemotherapeutic, a toxin, and combinations thereof.

Chemotherapeutics of the present invention are well known to those of skill in the art. Chemotherapeutics may include cell toxic substances such as DNA damaging agents, antimetabolites, anti-microtubule agents, antibiotic agents, etc. DNA damaging agents include alkylating agents, platinum-based agents, intercalating agents, and inhibitors of DNA replication. Non-limiting examples of DNA alkylating agents include cyclophosphamide, mechlorethamine, uramustine, melphalan, chlorambucil, ifosfamide, carmustine, lomustine, streptozocin, busulfan, temozolomide, pharmaceutically acceptable salts thereof, prodrugs, and combinations thereof. Non-limiting examples of platinum-based agents include cisplatin, carboplatin, oxaliplatin, nedaplatin, satraplatin, triplatin tetranitrate, pharmaceutically acceptable salts thereof, prodrugs, and combinations thereof. Non-limiting examples of intercalating agents include doxorubicin, daunorubicin, idarubicin, mitoxantrone, pharmaceutically acceptable salts thereof, prodrugs, and combinations thereof. Non-limiting examples of inhibitors of DNA replication include irinotecan, topotecan, amsacrine, etoposide, etoposide phosphate, teniposide, pharmaceutically acceptable salts thereof, prodrugs, and combinations thereof. Antimetabolites include folate antagonists such as methotrexate and premetrexed, purine antagonists such as 6-mercaptopurine, dacarbazine, and fludarabine, and pyrimidine antagonists such as 5-fluorouracil, arabinosylcytosine, capecitabine, gemcitabine, decitabine, pharmaceutically acceptable salts thereof, prodrugs, and combinations thereof. Anti-microtubule agents include without limitation vinca alkaloids, paclitaxel (Taxol®), docetaxel (Taxotere®), and ixabepilone (Ixempra®). Antibiotic agents include, without limitation, actinomycin, anthracyclines, valrubicin, epirubicin, bleomycin, plicamycin, mitomycin, gramicidin D, pharmaceutically acceptable salts thereof, prodrugs, and combinations thereof.

In the present invention, the term “toxin” means a poison or venom, preferably of plant or animal origin. Non-limiting examples of a toxin include diphtheria toxin or portions thereof, cytochalasin B, maytansinoid toxin, and auristatin toxin.

Preferably, the effector agent is a cell toxic substance selected from the group consisting of taxol, cytochalasin B, gramicidin D, ethidium bromide, emetine, mitomycin, etopside, tenopside, vincristine, vinblastine, colchicin, doxorubicin, daunorubicin, dihydroxy antracin dione, mitoxantrone, mithramycin, actinomycin D, 1-dehydrotestosterone, glycocorticoids, procaine, tetracaine, lidokaine, propranolol, puromycin and combinations thereof.

Preferably, the isolated mAb or antigen binding fragment thereof is conjugated to the effector agent or to the detectable marker by a linker.

As used herein, a “linker” is a chemical composition (including peptides) that physically connects one chemical species, e.g., the effector agent to another chemical species, e.g., an antibody or a fragment thereof. In the context of the present invention, a linker may be a cleavable linker or a non-cleavable linker. Generally, a cleavable linker may allow the two connected species to be separated upon exposure to a cleaving agent, such as cathepsin or an acid, with the ability to break at least one of the bonds in the linker, thereby separating the composition into at least two parts. Preferably, the linker is a cathepsin-cleavable linker. Non-cleavable linkers do not allow for the separation of chemical species connected by the linker. Other suitable, non-limiting examples of linkers include 12-(Boc-aminooxy)-dodecanoic acid, 12-(Boc-aminooxy)-lauric acid, Boc-AOAc-OH, 2-(Boc-aminooxy)-acetic acid, N-Boc-(carboxymethoxy)-amine, 3-maleimido-benzoic acid, 3-(2,5-Dioxo-2,5-dihydro-pyrrol-1-yl)benzoic acid, 3-maleimido-benzoic acid-OSu, MBS, N-Hydroxy-succinimidyl 3-maleimido-benzoate, ε-Maleimidocaproic acid-(2-nitro-4-sulfo)-phenyl ester, mal-sac-HNSA, 3-Maleimido-propionic acid, 3-(2,5-Dioxo-2,5-dihydro-pyrrol-1-yl)-propionic acid, MPA-OH, Maleoyl-β-Ala-OH, 3-(2-Pyridyldithio)-propionic acid-OSu, 3-(pyridin-2-yldisulfanyl)-propionic acid-OSu, and sulfo-N-succinimidyl 4-maleim idobutyrate.

Another embodiment of the present invention is an isolated mAb. The isolated mAb is selected from the group consisting of those clones listed in Table 1A below and having the listed characteristics:

TABLE 1A

| | Clone | Clone | Clone | | |
|---|---|---|---|---|---|
| | binding to | binding to | binding to | | |
| | CHO-TEM8 | HEK-TEM8 | HEK | | |
| Clone | (MFI) | (MFI) | (MFI) | Heavy | Light |
| 1A2.B12 | 1445 | 365 | 161 | IgG2a | K |
| 1A2.D12 | 1166 | 750 | 69 | IgG1 | K |
| 1A2.E12 | 813 | 486 | 127 | IgG1 | K |
| 1C2.A11 | 1527 | 593 | 74 | IgG1 | K |
| 1C2.B11 | 1427 | 472 | 35 | IgG1 | K |
| 1C2.C10 | 2605 | 603 | 55 | IgG1 | K |
| 1C2.E8 | 858 | 449 | 165 | IgG1 | K |
| 3C5.A11 | 1474 | 406 | 129 | IgG2b | K |
| 3C5.B10 | 1025 | 259 | 57 | IgG2b | K |
| 6H6.C12 | 767 | 257 | 105 | IgG1 | K |
| 7B2.A11 | 1241 | 331 | 90 | IgG2a | K |
| 7B2.B10 | 1376 | 243 | 82 | IgG2b | K |
| 7B7.B12 | 1411 | 432 | 142 | IgG2b | K |
| 8D3.D11 | 172 | 223 | 43 | IgG2b | K |
| 8H2.B11 | 1055 | 150 | 50 | IgG1 | K |
| 8H2.C12 | 2237 | 405 | 74 | IgG1 | K |

A further embodiment of the present invention is a chimeric antigen receptor (CAR). The CAR comprises:

(a) an antigen binding fragment of an antibody according to the present invention, particularly those identified in Table 1A; and

(b) a signaling domain of a T-cell receptor.

Chimeric antigen receptors provide cells in which they are expressed with additional specificity and functionality. For example, an antigen binding fragment of any antibody disclosed herein may specifically bind only to certain cell types that express the antigen, i.e., cancer cells. Expression of a CAR comprising this antigen binding fragment in an immune cell, i.e., a T-cell, will allow the aforementioned signaling domain of a T-cell receptor to generate an activating signal upon binding of the antigen binding fragment of the CAR to an antigen presenting cancer cell, thus mounting an immune response against a specific cell type.

Additionally, the immune response may be customized by varying the type of T-cell receptor linked to the antigen binding fragment of the CAR. In one aspect of this embodiment, the signaling domain of the T-cell receptor is selected from the group consisting of: (i) human CD28, human 4-1BB, and human CD3 intracellular T cell receptor signaling domains; (ii) human CD28 and human CD3ζ intracellular T cell receptor signaling domains; (iii) mouse CD28, mouse 4-1 BB, and mouse CD3ζ intracellular T cell receptor signaling domains; and (iv) mouse CD28 and mouse CD3ζ intracellular signaling domains.

An additional embodiment of the present invention is a modified antibody that binds a TEM8 antigen. The modified antibody comprises a variant human IgG1 Fc region, wherein the variant human IgG1 Fc region comprises at least one amino acid modification relative to the human IgG1 Fc region of a parent antibody disclosed herein that binds the TEM8 antigen, the amino acid modification(s) comprising amino acid modification(s) that alter the affinity or avidity of the variant Fc region for binding to an FcγR, such that the modified antibody exhibits, in an in vitro assay, enhanced effector function activity mediated by the FcγR binding in cells positive for the TEM8 antigen, and the parent antibody exhibits lower or non-detectable effector function activity in the cells using the in vitro assay, the amino acid modification(s) that alter the affinity or avidity of the variant Fc region for binding to an FcγR consisting of the modification of 1, 2, 3, 4 or 5 amino acid residues of the IgG1 Fc region of the parent antibody.

As used herein, “affinity” refers to the strength of noncovalent interactions between a single binding site of a molecule (e.g., an antibody) and its binding partner (e.g., an antigen or an Fc receptor). Unless indicated otherwise, as used herein, “binding affinity” refers to intrinsic binding affinity which reflects a 1:1 interaction between members of a binding pair (e.g., antibody/Fc receptor or antibody and antigen). The affinity of a molecule X for its partner Y can generally be represented by the dissociation constant (Kd). Affinity can be measured by common methods known in the art, including those described herein. For example, affinity may be measured by a BIAcore assay in which the Fc receptor is bound to a surface and binding of the variant is measured by Surface Plasmon Resonance (SPR). “Avidity” refers to the accumulated strength of multiple affinities of individual non-covalent binding interactions, such as between an antibody and a Fc receptor.

FcγRs (“Fc gamma receptors”) are well known to those of skill in the art and are generally known as those receptors typically found on immune cells that bind the Fc region of IgGs. FcγRs include FcγRI, FcγRIIa, FcγRIIb, FcγRIIIa, and FcγRIIIb. FcγRs exist on particular cells of the immune system, bind to particular IgG isotypes, can be activating or inhibiting, and exhibit varying levels of affinity for Fc regions. FcγRI mediates phagocytosis of target cells and binds IgG1 and IgG3 with high affinity on neutrophils and macrophages. FcγRIIa is activating, with low affinity, and binds IgG1, IgG2, and IgG3. FcγRIIb is inhibitory and may dampen FcγRI signaling. FcγRIIIa is activating, with low affinity, and controls antibody-dependent cell-mediated cell cytotoxicity (ADCC, discussed further below) and is expressed on various immune cells, including natural killer cells. FcγRIIIb is found in neutrophils and may control neutrophil activation. (Seidel, et al., 2013).

As used herein, “effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis (ADCP); resetting, opsonization, cell binding, down regulation of cell surface receptors (e.g. B cell receptor); and B cell activation.

Phagocytosis is the process of an immune cell engulfing and destroying a solid particle such as a bacterium or a dead cell. Briefly, phagocytosis involves a few general steps: recognition of a foreign particle, engulfment of the particle (similar to endocytosis), and degradation of the particle by proteolytic enzymes and reactive oxygen species. Recognition of a foreign particle can be accomplished by, for example, recognition of a foreign antigen by host antibodies. See Flannagan, et al., 2012 for a comprehensive review of phagocytosis. Assays for phagocytosis are known in the art, and kits for such assays are commercially available from e.g., Life Technologies.

Opsonization is the process of opsonins binding to substances in a body, thereby marking them for destruction via processes such as phagocytosis or antibody-dependent cellular cytotoxicity. Opsonins (e.g. antibodies, C3b) can be any substance that binds to a particle that causes recruitment of phagocytes or other immune system components to the particle, thereby marking the particles for destruction. Opsonization assays are disclosed in e.g., Burton et al., 2006.

Cell binding refers to antibodies or antibody fragments of the present invention causing the recruitment of cells of the immune system to bind to cells, such as those bearing a TEM8 antigen on their surface. Antibodies or antibody fragments of the present invention may bind to TEM8 antigens expressed on, for example, tumor cells, which may result in the recruitment of immune cells to, for example, the Fc region of the antibody or antibody fragment, thereby facilitating interaction of the tumor cell with the immune cell. Assays for cell binding are known in the art, and kits for such assays are commercially available from, e.g., Millipore.

Rosetting involves a central cell, for example, a tumor cell, and multiple cells that can bind to the central cell, for example, T cells expressing or simply comprising a TEM8 antibody or antigen binding fragment on their surface. The central cell (the tumor cell) may express TEM8, which allows anti-TEM8 antibody expressing T cells to bind to the central cell. The result is a central cell bound on all sides by antibody or antigen-binding fragment expressing cells, the whole of which may appear to be flowerlike and is detectable under a microscope.

Complement dependent cell mediated cytotoxicity (CDC) refers to cell killing effects or functions that are mediated via the complement system. The complement system is a highly complex component of the immune system that acts as a general surveillance mechanism for foreign substances (e.g. bacteria) or substances that would optimally be removed from the body (e.g. cell debris). Briefly, when a foreign substance is detected by the immune system by, for example, an antibody binding to the substance, components of the complement system may recognize this binding and stimulate cleavage of various pro-proteins of the complement system, thereby initiating a signaling cascade that can result in phagocytosis or lysis of the foreign substance or cause local inflammation. The multiple cleavage products that initiate the complement system response form an enzyme complex, C3 convertase, that can cleave C3, a normally inactive plasma protein, into C3a and C3b. C3b covalently binds to the surface of foreign substances, having an opsonization effect since C3b binding may induce downstream phagocytosis of the foreign substance. Alternatively, once C3 has been locally depleted, another plasma protein, C5, begins to be processed by C3 convertase, leading to the formation of a lytic pore in the membrane of the foreign substance and eventual destruction of the foreign substance. Complement dependent cell mediated cytotoxicity thus activates or otherwise utilizes the complement system in the destruction of a target cell (e.g. a cancer cell). See Ricklin, et al., 2010 for a more detailed description of the complement system. Assays for CDC are known in the art, and kits for such assays are commercially available from e.g., Cell Technology, Inc.

Antibody dependent cell-mediated cell cytotoxicity (ADCC) refers to the immune system process in which an antibody or antigen-binding fragment bound to a target cell is recognized by cells of the immune system such as, but not limited to, natural killer cells, macrophages, neutrophils, and eosinophils, thereby triggering the release of cell-permeable agents that induce target cell death. See Seidel, et al., 2013 for a more detailed description of ADCC. Assays for ADCC are known in the art, and kits for such assays are commercially available from e.g., Promega.

Another embodiment of the present invention is a modified antibody that binds a TEM8 antigen. The modified antibody comprises a variant human IgG1 Fc region, wherein the variant human IgG1 Fc region comprises at least one amino acid modification relative to the human IgG1 Fc region of a parent antibody disclosed herein that binds the TEM8 antigen, the amino acid modification(s) comprising amino acid modification(s) that alter the affinity or avidity of the variant Fc region for binding to an FcγR, such that the modified antibody exhibits enhanced effector function activity mediated by the FcγR binding in cells positive for the antigen and the parent antibody exhibits lower or non-detectable effector function activity; such that the modified antibody is therapeutically effective in a subject refractory to treatment with the parent antibody, the amino acid modification(s) that alter the affinity or avidity of the variant Fc region for binding to an FcγR consisting of the modification of 1, 2, 3, 4 or 5 amino acid residues of the IgG1 Fc region of the parent antibody. The effector functions according to this embodiment are as set forth above.

The term “refractory” means that the agent that is being administered to the patient has reduced efficacy in treating the disease compared to the same patient prior to becoming resistant to the agent.

As used herein, a “subject” is a mammal, preferably, a human. In addition to humans, categories of mammals within the scope of the present invention include, for example, agricultural animals, domestic animals, laboratory animals, etc. Some examples of agricultural animals include cows, pigs, horses, goats, etc. Some examples of domestic animals include dogs, cats, etc. Some examples of laboratory animals include primates, rats, mice, rabbits, guinea pigs, etc.

In one aspect of this embodiment, the modified antibody exhibits, in an in vitro assay, detectable effector function activity in cells derived from the subject, which cells are positive for the TEM8 antigen, and the parent antibody does not exhibit detectable functional activity in the cells using the in vitro assay. Representative non-limiting examples of such an in vitro assay include those disclosed in Lazar et al., 2006 and Moore et al., 2010.

In another aspect of the present invention, the TEM8 antigen is expressed on the surface of an endothelial cell. In the context of the present invention, the phrase “expressed on the surface of an endothelial cell” means that the TEM8 antigen may be present in whole or in part on the inner or outer surface of an endothelial cell. Preferably, the TEM8 antigen is located exclusively, or at least predominantly, on tumor endothelial cells. Some regions of an antigen may be embedded in the membrane and some regions of the antigen may be found on the intracellular surface of the endothelial cell membrane or on the extracellular surface of the endothelial cell membrane. In general, the location of a TEM8 antigen on a cell surface is well understood by those of skill in the art.

In the present invention, the amino acid modification(s) that alter the affinity or avidity of the variant Fc region for binding to an FcγR may comprise a substitution:

(i) at position 370 with glutamic acid, at position 396 with leucine and at position 270 with glutamic acid;

(ii) at position 419 with histidine, at position 396 with leucine and at position 270 with glutamic acid;

(iii) at position 240 with alanine, at position 396 with leucine and at position 270 with glutamic acid;

(iv) at position 255 with leucine, at position 396 with leucine and position 270 with glutamic acid;

(v) at position 255 with leucine, at position 396 with leucine, at position 270 with glutamic acid and at position 292 glycine; or

(vi) at position 255 with leucine, at position 396 with leucine, at position 270 with glutamic acid and at position 300 leucine.

In the present invention, at least one of the amino acid modification(s) that alter the affinity or avidity of the variant Fc region for binding to an FcγR may be in a CH2 domain of the variant human IgG1 Fc region. Preferably, the amino acid modification(s) that alter the affinity or avidity of the variant Fc region for binding to an FcγR in the CH2 domain comprises a substitution at position 240, 243, 247, 255, 270, 292, or 300 with another amino acid at that position.

In the present invention, at least one of the amino acid modification(s) that alter the affinity or avidity of the variant Fc region for binding to an FcγR may be a CH3 domain of the variant human IgG1 Fc region. Preferably, the amino acid modification(s) that alter the affinity or avidity of the variant Fc region for binding to an FcγR in the CH3 domain comprises a substitution at position 370, 392, 396, 419, or 421 with another amino acid at that position.

In the present invention, the amino acid modification(s) that alter the affinity or avidity of the variant Fc region for binding to an FcγR may also comprise at least one amino acid modification in the CH2 domain and at least one amino acid modification in the CH3 domain of the Fc region.

In the present invention, the amino acid modification(s) that alter the affinity or avidity of the variant Fc region for binding to an FcγR may further be in the hinge region of the human IgG1 heavy chain. Preferably, the modified antibody comprises at least one amino acid modification that alters the affinity or avidity of the variant Fc region for binding to an FcγR in the hinge region of the human IgG1 heavy chain.

The variant IgG1 Fc region of the modified antibody according to the present invention may specifically bind FcγRIIB with a lower affinity than the parent antibody binds FcγRIIB. See, e.g., WO 2007/024249 A2 for additional information regarding affinity/avidity modifying substitutions in Fc regions for binding to FcγRs.

The variant IgG1 Fc region of the modified antibody according to the present invention may also specifically bind FcγRIIIA with a greater affinity than the parent antibody binds FcγRIIIA. Preferably, the variant IgG1 Fc region also specifically binds FcγRIIB with a lower affinity than the parent antibody binds FcγRIIB.

The variant IgG1 Fc region of the modified antibody according to the present invention may additionally specifically bind FcγRIIA with a greater affinity than the parent antibody binds FcγRIIA. Preferably, the variant IgG1 Fc region also specifically binds FcγRIIB with a lower affinity than the parent antibody binds FcγRIIB.

In the present invention, greater affinity means that the variant IgG1 Fc region specifically binds its FcγR target, e.g., FcγRIIIA, or FcγRIIA from about 2 to about 100 times or more, such as about 5 to about 50 times, including about 10 to about 20 times, compared to the parent antibody. In the present invention, lower affinity means that the variant IgG1 Fc region specifically binds its FcγR target, e.g., FcγRIIB, from about 0.01 to about 0.5 times or less, such as about 0.02 to about 0.2 times, including about 0.05 to about 0.1 times, compared to the parent antibody. The variant IgG1 Fc region of a modified antibody according to the present invention may also specifically bind FcγRIIB with a greater affinity than when the parent binds FcγRIIB.

In another aspect of the present invention, the modified antibody detectably binds endothelial cells positive for the TEM8 antigen, which antigen is expressed at a density of 200 to 1,000 molecules/cell on the cells.

As used herein, the term “detectably binds” refers to an antibody that, when interacting with a TEM8 antigen on an endothelial cell, emits a signal that is observable and/or recordable by sight or by any of a number of devices well known to those of skill in the art or is otherwise detectable using techniques known in the art. For example, an antibody of the present invention may be linked to a fluorescent marker that can be utilized in immunostaining protocols well known to those of skill in the art. If TEM8 is present, the marker can be observed under appropriate fluorescent light when bound to TEM8 either via a fluorescence detection system, such as fluorimeter, a spectrometer, or a fluorescence activated cell sorting (FACS) apparatus.

The isolated mAb or the modified antibody according to the present invention is preferably non-fucosylated or has reduced fucosylation in the Fc region compared to the parent monoclonal antibody. Non-fucosylated antibodies or antibodies with reduced fucosylation generally cause enhanced antibody-dependent cell-mediated cytotoxicity (ADCC) due to higher affinity binding to FcγRIIIa than fucosylated antibodies. See Yamane-Ohnuki et al., 2009.

A further embodiment of the present invention is a composition. The composition comprises an effector agent or a detectable marker, which agent or marker is conjugated to a mAb or antigen binding fragment thereof, wherein the mAb or antigen binding fragment thereof:

(a) binds to tumor endothelial marker 8 membrane (TEM8) antigen in its native form occurring on the surface of a tumor cell;

(b) may be internalized by a tumor cell;

(c) binds strongly to tumor cells but not or only minimally to cells which lack expression of TEM8; and

(d) is characterized in that the mean fluorescence intensity (MFI) of the mAb or an antigen binding fragment thereof against a mammalian cell line expressing TEM8 is at least two times higher, preferably at least three, four, five, six, seven, eight, nine or ten times higher than the MFI of the mAb or an antigen binding fragment thereof against the mammalian cell line not expressing TEM8 at antigen saturation.

Suitable and preferred characteristics of the mAb or antigen binding fragment according to this embodiment, such as the composition of the framework region, the form of the mAb or antigen binding fragment thereof, and the isotype, are as disclosed herein.

In one aspect of this embodiment, the effector agent is a chemotherapeutic agent. Preferably, the chemotherapeutic agent is 5-fluorouracil or irinotecan.

In a further aspect of this embodiment, the effector agent is an anti-angiogenic agent. As used herein, an “anti-angiogenic agent” means a substance that reduces or inhibits the growth of new blood vessels, such as, e.g., an inhibitor of vascular endothelial growth factor (VEGF) and an inhibitor of endothelial cell migration. Anti-angiogenic agents include without limitation 2-methoxyestradiol, angiostatin, bevacizumab, cartilage-derived angiogenesis inhibitory factor, endostatin, IFN-α, IL-12, itraconazole, linomide, platelet factor-4, prolactin, SU5416, suramin, tasquinimod, tecogalan, tetrathiomolybdate, thalidomide, thrombospondin, thrombospondin, TNP-470, ziv-aflibercept, pharmaceutically acceptable salts thereof, prodrugs, and combinations thereof.

In an additional aspect of this embodiment, the effector agent is a toxin. Preferably, the toxin is a maytansinoid toxin. As used herein, a “maytansinoid toxin” is a derivative of maytansine, which is a tubulin-binding, microtubule inhibitor and cytotoxic agent. For example, maytansinoid toxins of the present invention include, but are not limited to, DM1.

In another aspect of this embodiment, the toxin is an auristatin toxin. As used herein, an “auristatin toxin” is an anti-mitotic agent that blocks microtubule polymerization and therefore prevents cell division. Auristatin toxins of the present invention include, but are not limited to, Monomethyl Auristatin E (MMAE) or Monomethyl Auristatin F (MMAF).

In an additional aspect of this embodiment, the detectable marker is selected from the group consisting of a fluorescent marker, an enzymatic marker, a heavy metal, a radioactive marker and combinations thereof.

In another aspect of this embodiment, the isolated mAb or antigen binding fragment thereof is conjugated to the effector agent or the detectable marker by a linker. Suitable and preferred linkers in this embodiment are as disclosed herein.

An additional embodiment of the present invention is a pharmaceutical composition. The pharmaceutical composition comprises an effective amount of a composition of the present invention and a pharmaceutically acceptable carrier.

Another embodiment of the present invention is a pharmaceutical composition that comprises any isolated mAb or an antigen binding fragment thereof disclosed herein. In another embodiment of the present invention, a pharmaceutical composition includes an effective amount of a mAb according to the present invention together with a pharmaceutically acceptable carrier.

A further embodiment of the present invention is a pharmaceutical composition that comprises an effective amount of any CAR disclosed herein and a pharmaceutically acceptable carrier.

An additional embodiment of the present invention is a pharmaceutical composition that comprises any modified antibody disclosed herein and a pharmaceutically acceptable carrier.

Another embodiment of the present invention is a kit for treating or ameliorating the effects of a cancer in a subject. The kit comprises any isolated mAb or an antigen binding fragment thereof disclosed herein packaged in combination with instructions for its use.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. In particular, the methods and compositions of the present invention may be used to slow the development of disease symptoms or delay the onset of the disease or condition, or halt the progression of disease development. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population, may fail to respond or respond inadequately to treatment.

As used herein, the terms “ameliorate”, “ameliorating” and grammatical variations thereof mean to decrease the severity of the symptoms of a disease in a subject.

A further embodiment of the present invention is a kit for treating or ameliorating the effects of a cancer in a subject. The kit comprises any CAR disclosed herein packaged in combination with instructions for its use.

An additional embodiment of the present invention is a kit for treating or ameliorating the effects of a cancer in a subject. The kit comprises any modified antibody disclosed herein packaged in combination with instructions for its use.

Another embodiment of the present invention is a kit for treating or ameliorating the effects of a cancer in a subject. The kit comprises any composition or any pharmaceutical composition disclosed herein packaged in combination with instructions for its use.

An additional embodiment of the present invention is a kit for the detection of a tumor cell. The kit comprises any isolated mAb or an antigen binding fragment thereof disclosed herein packaged in combination with instructions for its use.

Another embodiment of the present invention is a kit for the detection of pathological angiogenesis in a subject. The kit comprises any isolated mAb or an antigen binding fragment thereof disclosed herein packaged in combination with instructions for its use.

The kits of the present invention may also include suitable storage containers, e.g., ampules, vials, tubes, etc., for each pharmaceutical composition and other reagents, e.g., buffers, balanced salt solutions, etc., for use in administering the pharmaceutical compositions to subjects. The pharmaceutical compositions and other reagents may be present in the kits in any convenient form, such as, e.g., in a solution or in a powder form. The kits may further include instructions for use of the pharmaceutical compositions.

The kits may further include a packaging container, optionally having one or more partitions for housing the pharmaceutical composition and other optional reagents.

A further embodiment of the present invention is a method for identifying tumor cells. The method comprises:

(a) contacting a cell to be identified with any isolated mAb or an antigen binding fragment thereof disclosed herein; and

(b) identifying those cells to which the mAb or antigen binding fragment thereof specifically binds, wherein those cells bound to the mAb or antigen binding fragment thereof are tumor cells.

As used herein, “contacting a cell” means bringing the mAb or antigen binding fragment thereof and optionally one or more additional therapeutic agents into close proximity to the cells in need of such modulation, either in vitro or in vivo. This may be accomplished using conventional techniques of drug delivery to the subject or in the in vitro situation by, e.g., providing the mAb or antigen binding fragment thereof and optionally other agents to a culture media in which the cells are located. In this embodiment, the additional agents include agents for detecting the specific binding, blocking agents, detection agents and other like agents known in the art for use in such methods. See, e.g., the review by Gan et al., 2013.

In one aspect of this embodiment, the tumor cell is an endothelial cell that expresses TEM8.

In an additional aspect of this embodiment, the method further comprises, prior to step (a), obtaining a sample from a subject suspected of having a cancer and carrying out steps (a) and (b) with the sample. In the present invention, samples include, but are not limited to, blood, plasma, urine, skin, saliva, and biopsies. Conventional methods for obtaining samples from a subject, e.g., a human patient, are known in the art and may be used in the present invention.

In another aspect of this embodiment, the sample is selected from the group consisting of blood, urine, spinal fluid, amniotic fluid, serum, plasma, gingival, cervicular fluid, lachrymal fluid, lymph, mammary gland secretions, mucus, saliva, semen, tears, vaginal secretions, and vitreous humor.

An additional embodiment of the present invention is a method for treating or ameliorating the effects of a disease in a subject. The method comprises administering to a subject in need thereof an effective amount of any isolated mAb or an antigen binding fragment thereof disclosed herein.

Preferably, the disease is a cancer that expresses TEM8.

Another embodiment of the present invention is a method for treating or ameliorating the effects of a disease in a subject. The method comprises administering to a subject in need thereof an effective amount of any CAR disclosed herein.

In one aspect of this embodiment, the disease is a cancer that expresses TEM8.

A further embodiment of the present invention is a method for treating or ameliorating the effects of a disease in a subject. The method comprises administering to a subject in need thereof an effective amount of any modified antibody disclosed herein.

In one aspect of this embodiment, the disease is a cancer that expresses TEM8.

An additional embodiment of the present invention is a method for treating or ameliorating the effects of a disease in a subject. The method comprises administering to a subject in need thereof an effective amount of any of the compositions or any of the pharmaceutical compositions disclosed herein.

In one aspect of these embodiments, the disease is a cancer that expresses TEM8.

A further embodiment of the present invention is a method of modulating the binding of an anthrax protective antigen to a cell. The method comprises: contacting the cell with an effective amount of any isolated mAb or an antigen binding fragment thereof disclosed herein to modulate the binding of the anthrax protective antigen to the cell.

Anthrax protective antigen is one of three proteins that make up anthrax toxin, the remaining two being edema factor and lethal factor. Anthrax protective antigen interacts with cell membranes, forming a pore that allows entry of edema factor and lethal factor into the cell, subsequently killing the cell.

As used herein, the terms “modulate”, “modulating”, “modulator” and grammatical variations thereof mean to change, such as increasing or decreasing, the binding of anthrax protective antigen to a cell.

In one aspect of this embodiment, the contacting is carried out in vitro. In another aspect of this embodiment, the contacting is carried out in vivo.

An additional embodiment of the present invention is a polyclonal antibody which:

(a) binds to tumor endothelial marker 8 (TEM8) membrane antigen in its native form occurring on the surface of a tumor cell;

(b) may be internalized by a tumor cell;

(c) binds strongly to tumor cells but not or only minimally to cells which lack expression of TEM8; and

(d) is characterized in that the mean fluorescence intensity (MFI) of the polyclonal antibody against a mammalian cell line expressing TEM8 is at least two times higher than the MFI of the polyclonal antibody against the mammalian cell line not expressing TEM8 at antigen saturation.

Suitable and preferred mammalian cell lines according to the present invention, such as CHO or HEK cell lines, are as disclosed herein. In one aspect of this embodiment, the MFI of the polyclonal antibody against the HEK cell line expressing TEM8 is at least three times, such as at least four, five, six, seven eight, nine or ten times higher than the MFI of the polyclonal antibody against a HEK cell line not expressing TEM8 at antigen saturation.

In another aspect of this embodiment, the polyclonal antibody is 3C5.E7, 6H6.B11, 7B2.D9, or 7B7.E9.

In a further aspect of this embodiment, the polyclonal antibody is an IgG.

In another aspect of this embodiment, the polyclonal antibody is conjugated to a label or to an effector agent, optionally via a linker. Suitable and preferred labels, effector agents, and linkers according to the present invention are disclosed herein.

A further embodiment of the present invention is a polyclonal antibody selected from the group consisting of those clones listed in Table 1B below and having the listed characteristics:

TABLE 1B

| | binding to | binding to | binding to | | |
|---|---|---|---|---|---|
| | CHO-TEM8 | HEK-TEM8 | HEK | | |
| Name | (MFI) | (MFI) | (MFI) | Heavy | Light |
| 3C5.E7 | 536 | 215 | 92 | IgG2b/IgG1 | K |
| 6H6.B11 | 963 | 232 | 96 | IgG1 | K |
| 7B2.D9 | 678 | 284 | 102 | IgG2a | K |
| 7B7.E9 | 852 | 324 | 96 | IgG2a | K/λ |

Another embodiment of the present invention is a composition comprising an effector agent or a detectable marker, which agent or marker is conjugated to a polyclonal antibody, wherein the polyclonal antibody:

(a) binds to tumor endothelial marker 8 (TEM8) membrane antigen in its native form occurring on the surface of a tumor cell;

(b) may be internalized by a tumor cell;

(c) binds strongly to tumor cells but not or only minimally to cells which lack expression of TEM8; and

(d) is characterized in that the mean fluorescence intensity (MFI) of the polyclonal antibody against a mammalian cell line expressing TEM8 is at least two times higher than the MFI of the polyclonal antibody against the mammalian cell line not expressing TEM8 at antigen saturation.

In another aspect of this embodiment, the polyclonal antibody is 3C5.E7, 6H6.B11, 7B2.D9, or 7B7.E9.

In a further aspect of this embodiment, the polyclonal antibody is an IgG.

In another aspect of this embodiment, the polyclonal antibody is conjugated to a label or to an effector agent, optionally via a linker. Suitable and preferred labels, effector agents, and linkers according to the present invention are disclosed herein.

An additional embodiment of the present invention is a pharmaceutical composition. This pharmaceutical composition comprises an effective amount of any polyclonal antibody according to the present invention and a pharmaceutically acceptable carrier.

A further embodiment of the present invention is a kit for treating or ameliorating the effects of a cancer in a subject. This kit comprises any polyclonal antibody or pharmaceutical composition according to the present invention packaged in combination with instructions for use.

Another embodiment of the present invention is a kit for the detection of a tumor cell. This kit comprises any polyclonal antibody according to the present invention packaged in combination with instructions for use.

A further embodiment of the present invention is a kit for the detection of pathological angiogenesis in a subject. This kit comprises any polyclonal antibody according to the present invention packaged in combination with instructions for use. The polyclonal antibody kits may include various storage containers and reagents and may be packaged as previously disclosed.

Another embodiment of the present invention is a method for identifying tumor cells. The method comprises:

(a) contacting a cell to be identified with any polyclonal antibody disclosed herein; and

(b) identifying those cells to which the polyclonal antibody specifically binds, wherein those cells bound to the polyclonal antibody are tumor cells.

The method may be carried out in vitro or in vivo.

In one aspect of this embodiment, the tumor cell is an endothelial cell that expresses TEM8.

In an additional aspect of this embodiment, the method further comprises, prior to step (a), obtaining a sample from a subject suspected of having a cancer and carrying out steps (a) and (b) with the sample. Suitable and preferred samples according to the present invention are as disclosed herein.

An additional embodiment of the present invention is a method for treating or ameliorating the effects of a disease in a subject. This method comprises administering to a subject in need thereof an effective amount of any polyclonal antibody or pharmaceutical composition according to the present invention.

In one aspect of this embodiment, the disease is a cancer that expresses TEM8.

A further embodiment of the present invention is a method of modulating the binding of an anthrax protective antigen to a cell. The method comprises: contacting the cell with an effective amount of a polyclonal disclosed herein to modulate the binding of the anthrax protective antigen to the cell.

In one aspect of this embodiment, the contacting is carried out in vitro. In another aspect of this embodiment, the contacting is carried out in vivo.

In the present invention, an “effective amount” or a “therapeutically effective amount” of an antibody or antigen binding fragment thereof, modified antibody, or CAR of the invention, including the compositions and pharmaceutical compositions containing same, is an amount of such material that is sufficient to effect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of an agent or composition, i.e., antibody or antigen binding fragment thereof, modified antibody or CAR, according to the invention will be that amount of the agent or composition, which is the lowest dose effective to produce the desired effect. The effective dose of an agent or composition of the present invention may be administered as two, three, four, five, six or more sub-doses, administered separately at appropriate intervals throughout the day.

A suitable, non-limiting example of a dosage of an antibody, antigen binding fragment, modified antibody or CAR thereof disclosed herein is from about 0.1 mg/kg to about 120 mg/kg per day, such as from about 0.1 mg/kg to about 120 mg/kg per day, 7.5 mg/kg per day to about 30 mg/kg per day, including from about 1 mg/kg to about 100 mg/kg per day. Other representative dosages of such agents include about 0.1 mg/kg, 0.5 mg/kg, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 6 mg/kg, 7 mg/kg, 7.5 mg/kg, 8 mg/kg, 9 mg/kg, 10 mg/kg, 12.5 mg/kg, 15 mg/kg, 17.5 mg/kg, 20 mg/kg, 25 mg/kg, 30 mg/kg, 40 mg/kg, 50 mg/kg, 60 mg/kg, 70 mg/kg, 80 mg/kg, 90 mg/kg, 100 mg/kg, 110 mg/kg, and 120 mg/kg per day. The effective dose of antibodies and antigen binding fragments thereof, modified antibodies, and CARs of the present invention may be administered as two, three, four, five, six or more sub-doses, administered separately at appropriate intervals throughout the day.

The antibodies, antigen binding fragments thereof, modified antibodies, or CARs, or pharmaceutical compositions containing same of the present invention may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, the antibodies, antigen binding fragments thereof, modified antibodies, or CARs or pharmaceutical compositions containing same of the present invention may be administered in conjunction with other treatments. The pharmaceutical compositions of the present invention may be encapsulated or otherwise protected against gastric or other secretions, if desired.

The pharmaceutical compositions of the invention may comprise one or more active ingredients, e.g. antibodies or antigen binding fragments thereof, modified antibodies, or CARs in admixture with one or more pharmaceutically-acceptable diluents or carriers and, optionally, one or more other compounds, drugs, ingredients and/or materials. Regardless of the route of administration selected, the agents/compounds of the present invention are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, Pa.).

Pharmaceutically acceptable diluents or carriers are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable diluent or carrier used in a pharmaceutical composition of the invention must be “acceptable” in the sense of being compatible with the other ingredients of the formulation and not injurious to the subject. Diluents or carriers suitable for a selected dosage form and intended route of administration are well known in the art, and acceptable diluents or carriers for a chosen dosage form and method of administration can be determined using ordinary skill in the art.

The pharmaceutical compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

The pharmaceutical compositions of the present invention suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable diluents or carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

The pharmaceutical compositions of the present invention for rectal or vaginal administration may be presented as a suppository, which may be prepared by mixing one or more active ingredient(s) with one or more suitable nonirritating diluents or carriers which are solid at room temperature, but liquid at body temperature and, therefore, will melt in the rectum or vaginal cavity and release the active compound. The pharmaceutical compositions of the present invention which are suitable for vaginal administration also include pessaries, tampons, creams, gels, pastes, foams or spray formulations containing such pharmaceutically-acceptable diluents or carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable diluent or carrier. The ointments, pastes, creams and gels may contain excipients. Powders and sprays may contain excipients and propellants.

The pharmaceutical compositions of the present invention suitable for parenteral administrations may comprise one or more agent(s)/compound(s), i.e., antibodies or antigen binding fragments thereof, modified antibodies or CARs of the present invention, in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These pharmaceutical compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., a pharmaceutical composition of the present invention), it is desirable to slow its absorption from subcutaneous or intramuscular injection. This may be accomplished by the use of a liquid suspension of crystalline or amorphous material having poor water solubility.

Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized, for example, by filtration through a bacterial-retaining filter.

The formulations may be present in unit-dose or multi-dose sealed containers, for example, ampules and vials, and may be stored in a lyophilized condition requiring only the addition of the sterile liquid diluent or carrier, for example water for injection, immediately prior to use. Extemporaneous injection solutions and suspensions may be prepared from sterile powders, granules and tablets of the type described above.

### ADDITIONAL DEFINITIONS

As used herein, terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymers.

The term “amino acid” means naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. An “amino acid analog” means compounds that have the same basic chemical structure as a naturally occurring amino acid, e.g., a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. An “amino acid mimetic” means a chemical compound that has a structure that is different from the general chemical structure of an amino acid, but that functions similarly to a naturally occurring amino acid.

“Nucleic acid” or “oligonucleotide” or “polynucleotide” used herein means at least two nucleotides covalently linked together. Many variants of a nucleic acid may be used for the same purpose as a given nucleic acid. Thus, a nucleic acid also encompasses substantially identical nucleic acids and complements thereof.

Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be synthesized as a single stranded molecule or expressed in a cell (in vitro or in vivo) using a synthetic gene. Nucleic acids may be obtained by chemical synthesis methods or by recombinant methods.

The nucleic acid may also be a RNA such as a mRNA, tRNA, short hairpin RNA (shRNA), short interfering RNA (sRNA), double-stranded RNA (dsRNA), transcriptional gene silencing RNA (ptgsRNA), Piwi-interacting RNA, pri-miRNA, pre-miRNA, micro-RNA (miRNA), or anti-miRNA, as described, e.g., in U.S. patent application Ser. Nos. 11/429,720, 11/384,049, 11/418,870, and 11/429,720 and Published International Application Nos. WO 2005/116250 and WO 2006/126040.

The nucleic acid may also be an aptamer, an intramer, or a spiegelmer. The term “aptamer” refers to a nucleic acid or oligonucleotide molecule that binds to a specific molecular target. Aptamers are derived from an in vitro evolutionary process (e.g., SELEX (Systematic Evolution of Ligands by EXponential Enrichment), disclosed in U.S. Pat. No. 5,270,163), which selects for target-specific aptamer sequences from large combinatorial libraries. Aptamer compositions may be double-stranded or single-stranded, and may include deoxyribonucleotides, ribonucleotides, nucleotide derivatives, or other nucleotide-like molecules. The nucleotide components of an aptamer may have modified sugar groups (e.g., the 2′—OH group of a ribonucleotide may be replaced by 2′-F or 2′-NH₂), which may improve a desired property, e.g., resistance to nucleases or longer lifetime in blood. Aptamers may be conjugated to other molecules, e.g., a high molecular weight carrier to slow clearance of the aptamer from the circulatory system. Aptamers may be specifically cross-linked to their cognate ligands, e.g., by photo-activation of a cross-linker (Brody, E. N. and L. Gold (2000) J. Biotechnol. 74:5-13).

The term “intramer” refers to an aptamer which is expressed in vivo. For example, a vaccinia virus-based RNA expression system has been used to express specific RNA aptamers at high levels in the cytoplasm of leukocytes (Blind, M. et al. (1999) Proc. Natl. Acad. Sci. USA 96:3606-3610).

The term “spiegelmer” refers to an aptamer which includes L-DNA, L-RNA, or other left-handed nucleotide derivatives or nucleotide-like molecules. Aptamers containing left-handed nucleotides are resistant to degradation by naturally occurring enzymes, which normally act on substrates containing right-handed nucleotides.

A nucleic acid will generally contain phosphodiester bonds, although nucleic acid analogs may be included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those disclosed in U.S. Pat. Nos. 5,235,033 and 5,034,506. Nucleic acids containing one or more non-naturally occurring or modified nucleotides are also included within the definition of nucleic acid. The modified nucleotide analog may be located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino)propyl uridine, 5-bromo uridine; adenosines and guanosines modified at the 8-position, e.g. 8-bromo guanosine; deaza nucleotides, e.g. 7-deaza-adenosine; 0- and N-alkylated nucleotides, e.g. N6-methyl adenosine are suitable. The 2′-OH-group may be replaced by a group selected from H, OR, R, halo, SH, SR, NH₂, NHR, NR₂ or CN, wherein R is C₁-C₆ alkyl, alkenyl or alkynyl and halo is F, Cl, Br or I. Modified nucleotides also include nucleotides conjugated with cholesterol through, e.g., a hydroxyprolinol linkage as disclosed in Krutzfeldt et al., Nature (Oct. 30, 2005), Soutschek et al., Nature 432:173-178 (2004), and U.S. Patent Application Publication No. 20050107325. Modified nucleotides and nucleic acids may also include locked nucleic acids (LNA), as disclosed in U.S. Patent Application Publication No. 20020115080. Additional modified nucleotides and nucleic acids are disclosed in U.S. Patent Application Publication No. 20050182005. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made.

The terminology used herein is for the purpose of describing particular embodiments only and is not intended to be limiting. As used in the specification and the appended claims, the singular forms “a,” “an,” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

The following examples are provided to further illustrate the methods of the present invention. These examples are illustrative only and are not intended to limit the scope of the invention in any way.

### EXAMPLES

### Example 1

### Materials and Methods

### Generation and Characterization of Monoclonal Antibodies Specific to Human Tumor Endothelial Marker 8 (TEM8)

In general, monoclonal antibodies specific to human Tumor Endothelial Marker 8 (hTEM8) were generated at Abpro (Lexington, Mass.) using the mouse hybridoma technology. Briefly, HTP™ mice from Abpro were immunized with HEK293 cells expressing hTEM8 (HEK293-hTEM8) and subsequent fusion of hTEM8-specific B cells from immunized mouse lymph nodes with NSO myeloma fusion partner cells.

Construction of TEM8 and CMG2 vectors

A pcDNA3.1(+)-hTEM8 vector encoding the full length human TEM8 protein (GS50782 pcDNA3.1(+)-ANTXR1, SEQ ID NO: 7) was constructed at Epoch Life Science (Sugarland, TX) by cloning a synthesized human TEM8 into HindIII, XhoI digested pcDNA3.1(+) vector (Life Technologies, Carlsbad, Calif.). A pcDNA3.1(+)-hCMG2 vector encoding the full length human CMG2 protein (GS50831 pcDNA3.1(+)-CMG2, SEQ ID NO: 8) was similarly constructed by cloning the synthesized human CMG2 into HindIII, XhoI digested pcDNA3.1(+) vector (Life Technologies). Both constructs were sequenced and verified to be mutation-free.

### Cell Lines

HEK293 (293) and CHO cells were obtained from the American Type Culture Collection (ATCC). HEK293 cells were cultured in DMEM supplemented with 10% FBS. CHO cells were maintained in Ham's F12 medium supplemented with 10% fetal bovine serum (FBS). HEK293-hTEM8, HEK293-hCMG2 and CHO-hTEM8 cell lines were generated at Antibody Solutions (Sunnyvale, Calif.) by Metafectene (Biontex) mediated stable transfection of cells with a pcDNA3.1(+)-TEM8 or a pcDNA3.1(+)-CMG2 vector. The G418-resistant transfected cells were incubated with Protective Antigen (PA)-FITC conjugate (List Biological Laboratories, Campbell, Calif.) on ice for 30 minutes, and cells with high fluorescence intensity were sorted into wells of a 96-well plate using a fluorescence-activated cell sorter. The sorted clones were expanded, and evaluated for hTEM8 or hCMG2 expression based on PA-FITC binding. Cell lines expressing high levels of TEM8 or CMG2 were selected for immunization and/or screening purposes.

### Hybridoma Generation

### Phase I: Immunization

Five female HTP™ mice were immunized with HEK293-hTEM8 cells emulsified in Freund's Complete Adjuvant using the HTP™ immunization protocol (Abpro). Eight injections (5×106 HEK293-hTEM8/injection/mouse) were given over 3 weeks. Antibody serum titers were determined after the eighth immunization by Fluorescence Activated Cell Sorting (FACS).

For FACS, 50 μl of HEK293-hTEM8 cells (2.5×10⁵ in 96-well plates) were incubated for 1 hour on ice with 100 μl of post-immune mouse serum serially diluted in PBS containing 1% fetal bovine serum (Gibco cat#26140-079, Life Technologies). Cells were washed with 100 μl of ice cold 1% FBS in PBS. 50 μl of Alexa Fluor 488 goat anti-mouse IgG (Invitrogen Cat# A21235) (1:10000 dilution in 1% FBS PBS) was added, and the mixture was incubated for 1 hour on ice in the dark. After washing with ice-cold 1% FBS in PBS, cells were analyzed on a BD LSRII flow cytometer (Becton Dickinson, Franklin Lakes, N.J.). Live cells were gated and Alexa Fluor 488 MFI was analyzed.

### Phase II: Fusion

Lymph nodes were removed from two freshly euthanized mice that produced the highest antibody titers recognizing cell surface TEM8. The lymph nodes were washed with 5 ml RPMI (Gibco cat#11875-085, Life Technologies), dissected, and mashed over a 70 micron filter using a serological pipette. Isolated lymphocytes were washed through a cell strainer using 10 ml of RPMI media into a 50 ml conical tube. Cells were centrifuged at 1000 rpm for 5 minutes, re-suspended in 2 ml plain RPMI media, and incubated with 25 μl of Pronase (stock 10 mg/ml, EMD4Biosciences, Cat#537088, Life Technologies) for 2 minutes at room temperature. The reaction was terminated by addition of 0.5 ml FBS.

Equal amounts of freshly harvested lymphocytes (2×10⁷) and NSO myeloma fusion partner cells (ATCC Cat# PTA-4796) were mixed in RPMI medium and co-pelleted by centrifugation. Cells were washed twice with cytofusion medium (BTX Harvard Apparatus, Cat#47-0001, Holliston, Mass.), centrifuged, and re-suspended to 2×10⁷ cells/ml with cytofusion medium. Two milliliters of cell mixture were loaded to covered sterile microslides and placed under a microscope with ECM2001 for electrofusion at the designated conditions (AC 35 V RMS, 30 sec., post AC 9 sec.; Pulse length 10 uS, Voltage 3000, # of Pulse 1, # Repeats 0). Fused cells were left undisturbed for 5 minutes, and then carefully transferred to a tissue culture flask containing 85 ml of HAT (Sigma cat#25-046-CI, St. Louis, Mo.) selection medium, RPMI medium supplemented with 100 μM hypoxanthine, 0.4 μM aminopterin, 16 μM thymidine, 20% fetal bovine serum, 1×HFCS (Roche 11-363-735-001), 20% NCTC-109 (Gibco 21340-039), 5 mM L-glutamine, 1× Pen/Strep, 1 mM HEPES, and 0.1 mM nonessential amino acids. Cells were incubated for 20-30 minutes at 37° C., 8% CO₂-in-air, and 98% humidity in a CO₂ incubator. Cells were aliquotted into 96-well microtiter plates (100 μl/well, 8 plates/fusion) and grown to at least 50% confluence.

### Phase III: Screening of the Hybridoma Fusion Clones

CHO-hTEM8 cell flow cytometry was utilized in the primary fusion screen of hybridoma cells. 2×10⁴ CHO cells overexpressing hTEM8 (CHO-hTEM8) were incubated with hybridoma supernatant in sixteen 96 well fusion plates (8 plates per fusion), each well having a single concentration point, followed by staining with Alexa Fluor488 goat anti-mouse IgG. Live populations were gated and analyzed for Alexa Fluor 488 MFI. Diluted serum (1:400) from immunized mice was used as positive control. CHO parental line served as negative control.

Positive clones secreting hTEM8-specific monoclonal antibodies were expanded and retested by FACS with CHO-hTEM8 and HEK293-hTEM8 cells. HEK293-CMG2 cells were included as the counter-screen for TEM8. Parental CHO and HEK293 were used as negative controls.

### Phase IV: Subcloning, Screening, and Antibody Isotyping

Ten parental hybridomas assaying positive for hTEM8-specific antibody secretion (Table 3) were subcloned by limited dilution.

TABLE 3
Mean Fluorescence Intensities (MFIs) of Hybridoma Antibody Secretions by FACS

| Screening Cell | | | | | | | | | | | |
|---|---|---|---|---|---|---|---|---|---|---|---|
| Lines | | CHO- | | HEK- | HEK- | HEK, | | | | | |
| | Fusion | TEM8 | CHO | TEM8 | CMG 2 | 2^{nd} Ab | CHO | CHO-TEM8 | | | |
| Mouse | Clone* | No dil | No dil | No dil | No dil | No dil | ½ dil | ½ dil | 1/6 dil | 1/18 dil | 1/36 dil |
| Ms3588 | 1A2 | 325 | 24 | 232 | 121 | 63 | 9.3 | 784.3 | 645.8 | 555.4 | 339.0 |
| Ms3588 | 1C2 | 226 | 25.2 | 160 | 106 | 63 | 8.8 | 1067.1 | 944.7 | 745.0 | 392.8 |
| Ms3588 | 3C5 | 506 | 23.4 | 240 | 191 | 63 | 8.4 | 999.7 | 739.2 | 551.5 | 423.2 |
| Ms3588 | 6H4 | 503 | 20.6 | 588 | 301 | 63 | 8.1 | 607.3 | 294.6 | 135 | 65.2 |
| Ms3588 | 6H6 | 282 | 23.4 | 143 | 130 | 63 | 8.6 | 680.7 | 656.7 | 632.7 | 559.6 |
| Ms3571 | 7B2 | 238 | 23.8 | 124 | 51 | 63 | 14.1 | 492.4 | 518.1 | 486.0 | 455.4 |
| Ms3571 | 7B7 | 533 | 24.2 | 105 | 72 | 63 | 8.7 | 741.7 | 370.6 | 183.4 | 82.5 |
| Ms3571 | 8D3 | 487 | 24.4 | 886 | 133 | 63 | 10.3 | 437.6 | 93.7 | 25.1 | 17.3 |
| Ms3571 | 8H2 | 1185 | 22.4 | 384 | 152 | 63 | 7.7 | 935.4 | 399.3 | 201.7 | 71.1 |
| Ms3571 | 8G4 | 379 | 24.7 | 183 | 154 | 63 | 8.1 | 529.8 | 375.2 | 188.8 | 91.7 |

| Ms3588 serum as positive control | | | | |
|---|---|---|---|---|
| 66.7 | 704.7 | 480.1 | 206.4 | 99.4 |
| Diluent as background | | | | |
| 9.2 | 17.9 | 17.6 | 18.7 | 17.9 |

Note:
*These 10 fusion clones were selected based on their differential bindings to CHO-TEM8 and HEK-TEM8 cell lines vs. HEK-CMG2, CHO, and HEK parental lines. They were then serially diluted to generate subclones.

Hybridomas grown in 96-well plates were first transferred from each well to a separate well in 24-well plates. Cells were cultured overnight at 8% CO₂-in-air and 98% humidity in HAT supplemented Dulbecco's modified Eagle medium (DMEM, GIBCO#430-2100, Life Technologies) containing 100 μM hypoxanthine, 0.4 μM aminopterin, 16 μM thymidine, 20% FBS, 2 mM L-glutamine, 33.3 mM sodium bicarbonate, 20 mM HEPES, 1 mM sodium pyruvate, 0.1 mM nonessential amino acids penicillin (50 IU/ml), and streptomycin (50 μg/ml). Overnight cultures were subsequently diluted and seeded to 96-well plates with 0.8 cell/well (100 μl of 8-cells/ml dilution). Cells were grown to 10 to 50% confluence in the presence of mouse feeder cells. Cloning procedures were repeated until a stable and single hybridoma cell line was established. Hybridoma supernatants were assayed for hTEM8-specific antibody by FACS with CHO-hTEM8 and HEK293-hTEM8 cells. HEK293-CMG2 cells were included as the counter-screen for TEM8. Parental CHO and HEK293 were used as negative controls. Twenty subclones were selected and expanded for further characterization (Table 4).

Antibody isotypes were determined (Table 4) using a mouse monoclonal antibody isotyping kit (Sigma 1502-1 KT) following the manufacturer's instructions. Stock anti-isotype antibodies were diluted to 1:1000 in 1x PBS and added to ELISA plates at 50 μl/well for overnight incubation at 4° C. ELISA plates were blocked with 4% BSA in PBS (blocking buffer) for 1 hour at room temperature, followed by 3× washing with washing buffer (0.05% Tween-20 in PBS). Monoclonal hybridoma supernatant was added to the ELISA plate (50 μl/well) and incubated for 1 hour at RT. After 3× washing with washing buffer, 50 μl/well of HRP conjugated Goat-anti-mouse IgG-HRP (diluted 1:10,000 in Blocking Buffer) was added for a 1 hour incubation at room temperature, followed by incubation with 2,2′-azino-bis(3-ethylbenzthiazoline-6-sulphonic acid (ABTS, 50 μl/well) for 20 minutes at room temperature in the dark. The optical density was detected at 405 nm on an ELISA plate reader.

TABLE 4
MFIs of Subclone Antibody Secretions by FACS

| Subclone** | CHO-TEM8 | HEK-TEM8 | HEK | Heavy | Light |
|---|---|---|---|---|---|
| 1A2.B12 | 1445 | 365 | 161 | IgG2a | K |
| 1A2.D12 | 1166 | 750 | 69 | IgG1 | K |
| 1A2.E12 | 813 | 486 | 127 | IgG1 | K |
| 1C2.A11 | 1527 | 593 | 74 | IgG1 | K |
| 1C2.B11 | 1427 | 472 | 35 | IgG1 | K |
| 1C2.C10 | 2605 | 603 | 55 | IgG1 | K |
| 1C2.E8 | 858 | 449 | 165 | IgG1 | K |
| 3C5.A11 | 1474 | 406 | 129 | IgG2b | K |
| 3C5.B10 | 1025 | 259 | 57 | IgG2b | K |
| 3C5.E7*** | 536 | 215 | 92 | IgG2b/IgG1 | K |
| 6H6.B11*** | 963 | 232 | 96 | IgG1 | K |
| 6H6.C12 | 767 | 257 | 105 | IgG1 | K |
| 7B2.A11 | 1241 | 331 | 90 | IgG2a | K |
| 7B2.B10 | 1376 | 243 | 82 | IgG2b | K |
| 7B2.D9*** | 678 | 284 | 102 | IgG2a | K |
| 7B7.B12 | 1411 | 432 | 142 | IgG2b | K |
| 7B7.E9*** | 852 | 324 | 96 | IgG2a | K/λ |
| 8D3.D11 | 172 | 223 | 43 | IgG2b | K |
| 8H2.B11 | 1055 | 150 | 50 | IgG1 | K |
| 8H2.C12 | 2237 | 405 | 74 | IgG1 | K |

**These 20 subclones were selected based on their differential bindings to CHO-TEM8 and HEK-TEM8 cell lines vs. HEK-CMG2 and HEK parental lines.
***These subclones were determined to be polyclonal, requiring further subcloning in the future.

### DOCUMENTS

BURTON, Robert L. et al. “Development and validation of a fourfold multiplexed opsonization assay (MOPA4) for pneumococcal antibodies.” Clinical and vaccine immunology 13.9 (2006): 1004-1009.
FLANNAGAN, R. S., et al. (2012). The Cell Biology of Phagocytosis. Annual Review of Pathology: Mechanisms of Disease 7: 61-98.
GAN, Stephanie D et al. (2013) Enzyme Immunoassay and Enzyme-Linked Immunosorbent Assay. J Invest Dermatol 133 (9): e12.
LAZAR, Greg A., et al. “Engineered antibody Fc variants with enhanced effector function.” Proceedings of the National Academy of Sciences of the United States of America 103.11 (2006): 4005-4010.
KOENIG, S. and STAVENHAGEN, J. (2007). Engineering fc antibody regions to confer effector function. International Application No. WO 2007/024249 A2.
Moore, Gregory L., et al. “Engineered Fc variant antibodies with enhanced ability to recruit complement and mediate effector functions.” MAbs. Vol. 2. No. 2. 2010.
RICKLIN, D., et al. (2010). Complement: a key system for immune surveillance and homeostasis. Nature Immunology 11(9): 785-797.
SEIDEL, U. J. E., et al. (2013). Natural killer cell mediated antibody-dependent cellular cytotoxicity in tumor immunotherapy with therapeutic antibodies. Frontiers in Immunology 4: 1-8.
YAMANE-OHNUKI, N., et al. (2009). Production of therapeutic antibodies with controlled fucosylation. MAbs 1(3): 230-236.
YANG, M. Y., et al. (2011). The Cell Surface Structure of Tumor Endothelial Marker 8 (TEM8) is Regulated by the Actin Cytoskeleton. Biochim Biophys Acta. 1813(1): 39-49.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

1. An isolated monoclonal antibody (mAb) or an antigen binding fragment thereof, which:
(a) binds to tumor endothelial marker 8 (TEM8) membrane antigen in its native form occurring on the surface of a tumor cell;
(b) may be internalized by a tumor cell;
(c) binds strongly to tumor cells but not or only minimally to cells which lack expression of TEM8; and
(d) is characterized in that the mean fluorescence intensity (MFI) of the mAb or an antigen binding fragment thereof against a mammalian cell line expressing TEM8 is at least two times higher than the MFI of the mAb or an antigen binding fragment thereof against the mammalian cell line not expressing TEM8 at antigen saturation.

2. The isolated mAb or an antigen binding fragment thereof according to claim 1, wherein the mammalian cell line is a CHO cell line, and wherein the MFI of the mAb or an antigen binding fragment thereof against the CHO cell line expressing TEM8 is at least three times higher than the MFI of the mAb or an antigen binding fragment thereof against a CHO cell line not expressing TEM8 at antigen saturation.

3. The isolated mAb or an antigen binding fragment thereof according to claim 2, wherein the MFI of the mAb or an antigen binding fragment thereof against the CHO cell line expressing TEM8 is at least five times higher than the MFI of the mAb or an antigen binding fragment thereof against the CHO cell line not expressing TEM8 at antigen saturation.

4. The isolated mAb or an antigen binding fragment thereof according to claim 2, wherein the MFI of the mAb or an antigen binding fragment thereof against the CHO cell line expressing TEM8 is at least ten times higher than the MFI of the mAb or an antigen binding fragment thereof against the CHO cell line not expressing TEM8 at antigen saturation.

5. The isolated mAb or an antigen binding fragment thereof according to claim 1, which is selected from the group consisting of 1A2.B12, 1A2.D12, 1A2.E12, 1C2.A11, 1C2.B11, 1C2.C10, 1C2.E8, 3C5.A11, 3C5.B10, 6H6.C12, 7B2.A11, 7B2.B10, 7B7.B12, 8D3.D11, 8H2.B11, 8H2.C12, and antigen binding fragments thereof.

6. The isolated mAb or an antigen binding fragment thereof according to claim 1, which further comprises a human framework region.

7. The isolated mAb or an antigen binding fragment thereof according to claim 1, which is a humanized antibody, a chimeric antibody, or a recombinant antibody.

8. The isolated mAb or an antigen binding fragment thereof according to claim 1, wherein the antibody is an IgG.

9. The isolated mAb or an antigen binding fragment thereof according to claim 1, wherein the antigen binding fragment of the mAb is a Fv, a Fab, a F(ab′)2, a scFV or a scFV2 fragment.

10. The isolated mAb or an antigen binding fragment thereof according to claim 1, which is conjugated to a label or to an effector agent.

11. The isolated mAb or an antigen binding fragment thereof according to claim 10, wherein the label is selected from the group consisting of a fluorescent marker, an enzymatic marker, a heavy metal, a radioactive marker, and combinations thereof.

12. The isolated mAb or an antigen binding fragment thereof according to claim 10, wherein the effector agent is selected from the group consisting of a chemotherapeutic, a toxin, and combinations thereof.

13. The isolated mAb or an antigen binding fragment thereof according to claim 10, wherein the effector agent is a cell toxic substance selected from the group consisting of taxol, cytochalasin B, gramicidin D, ethidium bromide, emetine, mitomycin, etopside, tenopside, vincristine, vinblastine, colchicin, doxorubicin, daunorubicin, dihydroxy antracin dione, mitoxantrone, mithramycin, actinomycin D, 1-dehydrotestosterone, glycocorticoids, procaine, tetracaine, lidokaine, propranolol, puromycin and combinations thereof.

14. The isolated mAb or an antigen binding fragment thereof according to claim 10, wherein the isolated mAb or antigen binding fragment thereof is conjugated to the effector agent or to the detectable marker by a linker.

15. The isolated mAb or an antigen binding fragment thereof according to claim 14, wherein the linker is a cleavable linker.

16. The isolated mAb or an antigen binding fragment thereof according to claim 14, wherein the linker is a cathepsin-cleavable linker.

17. The isolated mAb or an antigen binding fragment thereof according to claim 14, wherein the linker is a non-cleavable linker.

18. An isolated mAb selected from the group consisting of those clones listed in Table 1A below and having the listed characteristics:
TABLE 1A

| | Clone | Clone | Clone | | |
|---|---|---|---|---|---|
| | binding to | binding to | binding to | | |
| | CHO-TEM8 | HEK-TEM8 | HEK | | |
| Clone | (MFI) | (MFI) | (MFI) | Heavy | Light |
| 1A2.B12 | 1445 | 365 | 161 | IgG2a | K |
| 1A2.D12 | 1166 | 750 | 69 | IgG1 | K |
| 1A2.E12 | 813 | 486 | 127 | IgG1 | K |
| 1C2.A11 | 1527 | 593 | 74 | IgG1 | K |
| 1C2.B11 | 1427 | 472 | 35 | IgG1 | K |
| 1C2.C10 | 2605 | 603 | 55 | IgG1 | K |
| 1C2.E8 | 858 | 449 | 165 | IgG1 | K |
| 3C5.A11 | 1474 | 406 | 129 | IgG2b | K |
| 3C5.B10 | 1025 | 259 | 57 | IgG2b | K |
| 6H6.C12 | 767 | 257 | 105 | IgG1 | K |
| 7B2.A11 | 1241 | 331 | 90 | IgG2a | K |
| 7B2.B10 | 1376 | 243 | 82 | IgG2b | K |
| 7B7.B12 | 1411 | 432 | 142 | IgG2b | K |
| 8D3.D11 | 172 | 223 | 43 | IgG2b | K |
| 8H2.B11 | 1055 | 150 | 50 | IgG1 | K |
| 8H2.C12 | 2237 | 405 | 74 | IgG1 | K |

19. A chimeric antigen receptor (CAR) comprising:
(a) an antigen binding fragment of an antibody according to claim 1; and
(b) a signaling domain of a T-cell receptor.

20. The CAR according to claim 19, wherein the signaling domain of the T-cell receptor is selected from the group consisting of: (i) human CD28, human 4-1 BB, and human CD3ζ intracellular T cell receptor signaling domains; (ii) human CD28 and human CD3ζ intracellular T cell receptor signaling domains; (iii) mouse CD28, mouse 4-1BB, and mouse CD3ζ intracellular T cell receptor signaling domains; and (iv) mouse CD28 and mouse CD3ζ intracellular signaling domains.

21. A modified antibody that binds a TEM8 antigen, the modified antibody comprising a variant human IgG1 Fc region, wherein the variant human IgG1 Fc region comprises at least one amino acid modification relative to the human IgG1 Fc region of a parent antibody according to claim 18 that binds the TEM8 antigen, the amino acid modification(s) comprising amino acid modification(s) that alter the affinity or avidity of the variant Fc region for binding to an FcγR, such that the modified antibody exhibits, in an in vitro assay, enhanced effector function activity mediated by the FcγR binding in cells positive for the TEM8 antigen, and the parent antibody exhibits lower or non-detectable effector function activity in the cells using the in vitro assay, the amino acid modification(s) that alter the affinity or avidity of the variant Fc region for binding to an FcγR consisting of the modification of 1, 2, 3, 4 or 5 amino acid residues of the IgG1 Fc region of the parent antibody.

22. A modified antibody that binds a TEM8 antigen, the modified antibody comprising a variant human IgG1 Fc region, wherein the variant human IgG1 Fc region comprises at least one amino acid modification relative to the human IgG1 Fc region of a parent antibody according to claim 18 that binds the TEM8 antigen, the amino acid modification(s) comprising amino acid modification(s) that alter the affinity or avidity of the variant Fc region for binding to an FcγR, such that the modified antibody exhibits enhanced effector function activity mediated by the FcγR binding in cells positive for the antigen and the parent antibody exhibits lower or non-detectable effector function activity; such that the modified antibody is therapeutically effective in a subject refractory to treatment with the parent antibody, the amino acid modification(s) that alter the affinity or avidity of the variant Fc region for binding to an FcγR consisting of the modification of 1, 2, 3, 4 or 5 amino acid residues of the IgG1 Fc region of the parent antibody.

23. The modified antibody of claim 22, wherein the modified antibody exhibits, in an in vitro assay, detectable effector function activity in cells derived from the subject, which cells are positive for the TEM8 antigen, and the parent antibody does not exhibit detectable functional activity in the cells using the in vitro assay.

24. The modified antibody according to claim 21, wherein the TEM8 antigen is expressed on the surface of an endothelial cell.

25. The modified antibody according to claim 21, wherein the amino acid modification(s) that alter the affinity or avidity of the variant Fc region for binding to an FcγR comprise(s) a substitution:
(i) at position 370 with glutamic acid, at position 396 with leucine and at position 270 with glutamic acid;
(ii) at position 419 with histidine, at position 396 with leucine and at position 270 with glutamic acid;
(iii) at position 240 with alanine, at position 396 with leucine and at position 270 with glutamic acid;
(iv) at position 255 with leucine, at position 396 with leucine and position 270 with glutamic acid;
(v) at position 255 with leucine, at position 396 with leucine, at position 270 with glutamic acid and at position 292 glycine; or
(vi) at position 255 with leucine, at position 396 with leucine, at position 270 with glutamic acid and at position 300 leucine.

26. The modified antibody according to claim 21, wherein at least one of the amino acid modification(s) that alter the affinity or avidity of the variant Fc region for binding to an FcγR is in a CH2 domain of the variant human IgG1 Fc region.

27. The modified antibody according to claim 26, wherein the amino acid modification(s) that alter the affinity or avidity of the variant Fc region for binding to an FcγR in the CH2 domain comprises a substitution at position 240, 243, 247, 255, 270, 292, or 300 with another amino acid at that position.

28. The modified antibody according to claim 21, wherein at least one of the amino acid modification(s) that alter the affinity or avidity of the variant Fc region for binding to an FcγR is in a CH3 domain of the variant human IgG1 Fc region.

29. The modified antibody according to claim 28, wherein the amino acid modification(s) that alter the affinity or avidity of the variant Fc region for binding to an FcγR in the CH3 domain comprises a substitution at position 370, 392, 396, 419, or 421 with another amino acid at that position.

30. The modified antibody according to claim 21, wherein the amino acid modification(s) that alter the affinity or avidity of the variant Fc region for binding to an FcγR comprises at least one amino acid modification in the CH2 domain and at least one amino acid modification in the CH3 domain of the Fc region.

31. The modified antibody according to claim 21, wherein the amino acid modification(s) that alter the affinity or avidity of the variant Fc region for binding to an FcγR is in the hinge region of the human IgG1 heavy chain.

32. The modified antibody according to claim 31 comprising at least one amino acid modification that alters the affinity or avidity of the variant Fc region for binding to an FcγR in the hinge region of the human IgG1 heavy chain.

33. The modified antibody according to claim 21, which variant IgG1 Fc region specifically binds FcγRIIIA with a greater affinity than the parent antibody binds FcγRIIIA.

34. The modified antibody according to claim 21, which variant IgG1 Fc region specifically binds FcγRIIA with a greater affinity than the parent antibody binds FcγRIIA.

35. The modified antibody according to claim 21, which variant IgG1 Fc region specifically binds FcγRIIB with a lower affinity than the parent antibody binds FcγRIIB.

36. The modified antibody according to claim 33, which variant IgG1 Fc region specifically binds FcγRIIB with a lower affinity than the parent antibody binds FcγRIIB.

37. The modified antibody according to claim 34, which variant IgG1 Fc region specifically binds FcγRIIB with a lower affinity than the parent antibody binds FcγRIIB.

38. The modified antibody according to claim 21, which detectably binds endothelial cells positive for the TEM8 antigen, which antigen is expressed at a density of 200 to 1,000 molecules/cell on the cells.

39. The modified antibody according to claim 21 wherein the effector function is antibody dependent cell-mediated cell cytotoxicity (ADCC).

40. The modified antibody according to claim 21 wherein the effector function is phagocytosis, opsonization, cell binding, rosetting, complement dependent cell mediated cytotoxicity (CDC), or antibody dependent cell-mediated cell cytotoxicity (ADCC).

41. An isolated mAb according to claim 18, wherein the antibody is non-fucosylated or has reduced fucosylation in the Fc region compared to the parent monoclonal antibody.

42. A modified antibody according to claim 21, wherein the antibody is non-fucosylated or has reduced fucosylation in the Fc region compared to the parent monoclonal antibody.

43. A composition comprising an effector agent or a detectable marker, which agent or marker is conjugated to a mAb or antigen binding fragment thereof, wherein the mAb or antigen binding fragment thereof:
(a) binds to tumor endothelial marker 8 membrane (TEM8) antigen in its native form occurring on the surface of a tumor cell;
(b) may be internalized by a tumor cell;
(c) binds strongly to tumor cells but not or only minimally to cells which lack expression of TEM8; and
(d) is characterized in that the mean fluorescence intensity (MFI) of the mAb or an antigen binding fragment thereof against a mammalian cell line expressing TEM8 is at least two times higher than the MFI of the mAb or an antigen binding fragment thereof against the mammalian cell line not expressing TEM8 at antigen saturation.

44. The composition according to claim 43, wherein the mAb or antigen binding fragment thereof, comprises a human framework region.

45. The composition according to claim 43, wherein the antibody is an IgG.

46. The composition according to claim 43, which comprises the antigen binding fragment of the mAb.

47. The composition according to claim 43, wherein the antigen binding fragment is a Fv, a Fab, a F(ab′)2, a scFV or a scFV2 fragment.

48. The composition according to claim 43, wherein the effector agent is a chemotherapeutic agent.

49. The composition according to claim 48, wherein the chemotherapeutic agent is 5-fluorouracil or irinotecan.

50. The composition according to claim 43, wherein the effector agent is an anti-angiogenic agent.

51. The composition according to claim 43, wherein the effector agent is a toxin.

52. The composition according to claim 51, wherein the toxin is a maytansinoid toxin.

53. The composition according to claim 52, wherein the maytansinoid toxin is DM1.

54. The composition according to claim 51, wherein the toxin is an auristatin toxin.

55. The composition according to claim 54, wherein the auristatin toxin is Monomethyl Auristatin E (MMAE) or Monomethyl Auristatin F (MMAF).

56. The composition according to claim 43, wherein the detectable marker is selected from the group consisting of a fluorescent marker, an enzymatic marker, a heavy metal, a radioactive marker and combinations thereof.

57. The composition according to claim 43, wherein the isolated mAb or antigen binding fragment thereof is conjugated to the effector agent or the detectable marker by a linker.

58. The composition according to claim 57, wherein the linker is a cleavable linker.

59. The composition according to claim 57, wherein the linker is a cathepsin-cleavable linker.

60. The composition according to claim 57, wherein the linker is a non-cleavable linker.

61. A pharmaceutical composition comprising an effective amount of the composition of claim 43 and a pharmaceutically acceptable carrier.

62. A pharmaceutical composition comprising an isolated mAb or an antigen binding fragment thereof according to claim 1.

63. A pharmaceutical composition comprising an effective amount of a mAb according to claim 18 and a pharmaceutically acceptable carrier.

64. A pharmaceutical composition comprising an effective amount of a CAR according to claim 19 and a pharmaceutically acceptable carrier.

65. A pharmaceutical composition comprising the modified antibody of any claim 21 and a pharmaceutically acceptable carrier.

66. A kit for treating or ameliorating the effects of a cancer in a subject comprising an isolated mAb or an antigen binding fragment thereof according to claim 1 packaged in combination with instructions for its use.

67. A kit for treating or ameliorating the effects of a cancer in a subject, the kit comprising the isolated mAb according to claim 18 packaged in combination with instructions for its use.

68. A kit for treating or ameliorating the effects of a cancer in a subject, the kit comprising the CAR according to claim 19 packaged in combination with instructions for its use.

69. A kit for treating or ameliorating the effects of a cancer in a subject, the kit comprising the modified antibody according to claim 21 packaged in combination with instructions for its use.

70. A kit for treating or ameliorating the effects of a cancer in a subject, the kit comprising the composition of claim 43 packaged in combination with instructions for its use.

71. A kit for treating or ameliorating the effects of a cancer in a subject comprising a pharmaceutical composition according to claim 61 packaged in combination with instructions for its use.

72. A kit for treating or ameliorating the effects of a cancer in a subject comprising a pharmaceutical composition according to claim 62 packaged in combination with instructions for its use.

73. A kit for treating or ameliorating the effects of a cancer in a subject comprising a pharmaceutical composition according to claim 63 packaged in combination with instructions for its use.

74. A kit for treating or ameliorating the effects of a cancer in a subject comprising a pharmaceutical composition according to claim 64 packaged in combination with instructions for its use.

75. A kit for treating or ameliorating the effects of a cancer in a subject comprising a pharmaceutical composition according to claim 65 packaged in combination with instructions for its use.

76. A kit for the detection of a tumor cell comprising an isolated mAb or an antigen binding fragment thereof according to claim 1 packaged in combination with instructions for its use.

77. A kit for the detection of a tumor cell comprising the isolated mAb according to claim 18 packaged in combination with instructions for its use.

78. A kit for the detection of pathological angiogenesis in a subject comprising an isolated mAb or an antigen binding fragment thereof according to claim 1 packaged in combination with instructions for its use.

79. A kit for the detection of pathological angiogenesis in a subject comprising the isolated mAb according to claim 18 packaged in combination with instructions for its use.

80. A method for identifying tumor cells comprising:
(a) contacting a cell to be identified with an isolated mAb or an antigen binding fragment thereof according to claim 1; and
(b) identifying those cells to which the mAb or antigen binding fragment thereof specifically binds, wherein those cells bound to the mAb or antigen binding fragment thereof are tumor cells.

81. The method according to claim 80, wherein the tumor cell is an endothelial cell that expresses TEM8.

82. The method according to claim 80, which is carried out in vitro.

83. The method according to claim 80, which is carried out in vivo.

84. The method according to claim 80 further comprising, prior to step (a), obtaining a sample from a subject suspected of having a cancer and carrying out steps (a) and (b) with the sample.

85. The method according to claim 80, wherein the sample is selected from the group consisting of blood, urine, spinal fluid, amniotic fluid, serum, plasma, gingival, cervicular fluid, lachrymal fluid, lymph, mammary gland secretions, mucus, saliva, semen, tears, vaginal secretions, and vitreous humor.

86. A method for treating or ameliorating the effects of a disease in a subject comprising administering to a subject in need thereof an effective amount of the isolated mAb or an antigen binding fragment thereof according to claim 1.

87. The method according to claim 86, wherein the disease is a cancer that expresses TEM8.

88. A method for treating or ameliorating the effects of a disease in a subject comprising administering to a subject in need thereof an effective amount of the isolated mAb according to claim 18.

89. The method according to claim 88, wherein the disease is a cancer that expresses TEM8.

90. A method for treating or ameliorating the effects of a disease in a subject comprising administering to a subject in need thereof an effective amount of the CAR according to claim 19.

91. The method according to claim 90, wherein the disease is a cancer that expresses TEM8.

92. A method for treating or ameliorating the effects of a disease in a subject comprising administering to a subject in need thereof an effective amount of the modified antibody according to claim 21.

93. The method according to claim 92, wherein the disease is a cancer that expresses TEM8.

94. A method for treating or ameliorating the effects of a disease in a subject comprising administering to a subject in need thereof an effective amount of the composition according to claim 43.

95. The method according to claim 94, wherein the disease is a cancer that expresses TEM8.

96. A method for treating or ameliorating the effects of a disease in a subject comprising administering to a subject in need thereof an effective amount of the pharmaceutical composition according to claim 61.

97. The method according to claim 96, wherein the disease is a cancer that expresses TEM8.

98. A method for treating or ameliorating the effects of a disease in a subject comprising administering to a subject in need thereof an effective amount of the pharmaceutical composition according to claim 62.

99. The method according to claim 98, wherein the disease is a cancer that expresses TEM8.

100. A method for treating or ameliorating the effects of a disease in a subject comprising administering to a subject in need thereof an effective amount of the pharmaceutical composition according to claim 63.

101. The method according to claim 100, wherein the disease is a cancer that expresses TEM8.

102. A method for treating or ameliorating the effects of a disease in a subject comprising administering to a subject in need thereof an effective amount of the pharmaceutical composition according to claim 64.

103. The method according to claim 102, wherein the disease is a cancer that expresses TEM8.

104. A method for treating or ameliorating the effects of a disease in a subject comprising administering to a subject in need thereof an effective amount of the pharmaceutical composition according to claim 65.

105. The method according to claim 104, wherein the disease is a cancer that expresses TEM8.

106. A method of modulating the binding of an anthrax protective antigen to a cell comprising: contacting the cell with an effective amount of an isolated mAb or an antigen binding fragment thereof according to claim 1 to modulate the binding of the anthrax protective antigen to the cell.

107. The method according to claim 106, wherein the contacting is carried out in vitro.

108. The method according to claim 106, wherein the contacting is carried out in vivo.

109. A method of modulating the binding of an anthrax protective antigen to a cell comprising: contacting the cell with an effective amount of an isolated mAb or an antigen binding fragment thereof according to claim 18 to modulate the binding of the anthrax protective antigen to the cell.

110. The method according to claim 109, wherein the contacting is carried out in vitro.

111. The method according to claim 109, wherein the contacting is carried out in vivo.

112. A polyclonal antibody which:
(a) binds to tumor endothelial marker 8 (TEM8) membrane antigen in its native form occurring on the surface of a tumor cell;
(b) may be internalized by a tumor cell;
(c) binds strongly to tumor cells but not or only minimally to cells which lack expression of TEM8; and
(d) is characterized in that the mean fluorescence intensity (MFI) of the polyclonal antibody against a mammalian cell line expressing TEM8 is at least two times higher than the MFI of the polyclonal antibody against the mammalian cell line not expressing TEM8 at antigen saturation.

113. A polyclonal antibody selected from the group consisting of those clones listed in Table 1B below and having the listed characteristics:
TABLE 1B

| | binding to | binding to | binding to | | |
|---|---|---|---|---|---|
| | CHO-TEM8 | HEK-TEM8 | HEK | | |
| Name | (MFI) | (MFI) | (MFI) | Heavy | Light |
| 3C5.E7 | 536 | 215 | 92 | IgG2b/IgG1 | K |
| 6H6.B11 | 963 | 232 | 96 | IgG1 | K |
| 7B2.D9 | 678 | 284 | 102 | IgG2a | K |
| 7B7.E9 | 852 | 324 | 96 | IgG2a | K/λ |

114. A composition comprising an effector agent or a detectable marker, which agent or marker is conjugated to a polyclonal antibody, wherein the polyclonal antibody:
(a) binds to tumor endothelial marker 8 (TEM8) membrane antigen in its native form occurring on the surface of a tumor cell;
(b) may be internalized by a tumor cell;
(c) binds strongly to tumor cells but not or only minimally to cells which lack expression of TEM8; and
(d) is characterized in that the mean fluorescence intensity (MFI) of the polyclonal antibody against a mammalian cell line expressing TEM8 is at least two times higher than the MFI of the polyclonal antibody against the mammalian cell line not expressing TEM8 at antigen saturation.

115. A pharmaceutical composition comprising an effective amount of a polyclonal antibody according to claim 112 and a pharmaceutically acceptable carrier.

116. A kit for treating or ameliorating the effects of a cancer in a subject comprising a polyclonal antibody according claim 112 packaged in combination with instructions for use.

117. A kit for treating or ameliorating the effects of a cancer in a subject comprising a pharmaceutical composition according to claim 115 packaged in combination with instructions for use.

118. A kit for the detection of a tumor cell comprising a polyclonal antibody according to claim 112 packaged in combination with instructions for use.

119. A method for treating or ameliorating the effects of a disease in a subject comprising administering to a subject in need thereof an effective amount of a polyclonal antibody according claim 112.


SEQUENCE LISTING (6 sequences)

SEQ ID NO: 1 - DNA, 5909 bp, Homo sapiens
atcatatttaaaatctgggacaaagaaccgtcgggacggaactccttccattgcaaaagctcggcgcggcctcgggagctgcccggcggccccggaccgaggcagccctcccctttaaaagaagcggaggacaggattgggatccttgaaacccgaaacccagaaacagcatcggagcggaaaccagaggggaaaccttgaactcctccagacaattgcttccggggagttgcgagggagcgagggggaataaaggacccgcgaggaagggcccgcggatggcgcgtccctgagggtcgtggcgagttcgcggagcgtgggaaggagcggaccctgctctccccgggctgcgggccatggccacggcggagcggagagccctcggcatcggcttccagtggctctctttggccactctggtgctcatctgcgccgggcaagggggacgcagggaggatgggggtccagcctgctacggcggatttgacctgtacttcattttggacaaatcaggaagtgtgctgcaccactggaatgaaatctattactttgtggaacagttggctcacaaattcatcagcccacagttgagaatgtcctttattgttttctccacccgaggaacaaccttaatgaaactgacagaagacagagaacaaatccgtcaaggcctagaagaactccagaaagttctgccaggaggagacacttacatgcatgaaggatttgaaagggccagtgagcagatttattatgaaaacagacaagggtacaggacagccagcgtcatcattgctttgactgatggagaactccatgaagatctctttttctattcagagagggaggctaataggtctcgagatcttggtgcaattgtttactgtgttggtgtgaaagatttcaatgagacacagctggcccggattgcggacagtaaggatcatgtgtttcccgtgaatgacggctttcaggctctgcaaggcatcatccactcaattttgaagaagtcctgcatcgaaattctagcagctgaaccatccaccatatgtgcaggagagtcatttcaagttgtcgtgagaggaaacggcttccgacatgcccgcaacgtggacagggtcctctgcagcttcaagatcaatgactcggtcacactcaatgagaagcccttttctgtggaagatacttatttactgtgtccagcgcctatcttaaaagaagttggcatgaaagctgcactccaggtcagcatgaacgatggcctctcttttatctccagttctgtcatcatcaccaccacacactgttctgacggttccatcctggccatcgccctgctgatcctgttcctgctcctagccctggctctcctctggtggttctggcccctctgctgcactgtgattatcaaggaggtccctccaccccctgccgaggagagtgaggaagaagatgatgatggtctgcctaagaaaaagtggccaacggtagacgcctcttattatggtgggagaggcgttggaggcattaaaagaatggaggttcgttggggagaaaagggctccacagaagaaggtgctaagttggaaaaggcaaagaatgcaagagtcaagatgccggagcaggaatatgaattccctgagccgcgaaatctcaacaacaatatgcgtcggccttcttccccccggaagtggtactctccaatcaagggaaaactcgatgccttgtgggtcctactgaggaaaggatatgatcgtgtgtctgtgatgcgtccacagccaggagacacggggcgctgcatcaacttcaccagggtcaagaacaaccagccagccaagtacccactcaacaacgcctaccacacctcctcgccgcctcctgcccccatctacactcccccacctcctgcgccccactgccctcccccgccccccagcgcccctacccctcccatcccgtccccaccttccacccttccccctcctccccaggctccacctcccaacagggcacctcctccctcccgccctcctccaaggccttctgtctagagcccaaagttcctgctctgggctctctcagaaacttcaggagatgttagaacaagtctttccagttagagaagaggagtggtgataaagcccactgaccttcacacattctaaaaattggttggcaatgccagtataccaacaatcatgatcagctgaaagaaacagatattttaaattgccagaaaacaaatgatgaggcaactacagtcagatttatagccagccatctatcacctctagaaggttccagagacagtgaaactgcaagatgctctcaacaggattatgtctcatggagaccagtaagaaaatcatttatctgaaggtgaaatgcagagttggataagaaatacattgctgggtttctaaaatgctgccttcctgcctctactccacctccatccctggactttggacccttggcctaggagcctaaggaccttcacccctgtgcaccacccaagaaagaggaaaactttgcctacaactttggaaatgctggggtccctggtgtggtaagaaactcaacatcagacgggtatgcagaaggatgttcttctgggatttgcaggtacataaaaaatgtatggcatcttttccttgcaaattcttccagtttccaagtgagaaggggagcaggtgtttactgatggaaaaggtatgttgctatgttgatgtgtaagtgaaatcagttgtgtgcaatagacaggggcgtattcatgggagcatcagccagtttctaaaacccacaggccatcagcagctagaggtggctggctttggccagacatggaccctaaatcaacagacaatggcattgtcgaagagcaacctgttaatgaatcatgttaaaaatcaaggtttggcttcagtttaaatcacttgaggtatgaagtttatcctgttttccagagataaacataagttgatcttcccaaaataccatcattaggacctatcacacaatatcactagttttttttgtttgtttgttttttgttttttttcttggtaaagccatgcaccacagacttctgggcagagctgagagacaatggtcctgacataataaggatctttgattaacccccataaggcatgtgtgtgtatacaaatatacttctctttggcttttcgacatagaacctcagctgttaaccaaggggaaatacatcagatctgcaacacagaaatgctctgcctgaaatttccaccatgcctaggactcaccccatttatccaggtctttctggatctgtttaatcaataagccctataatcacttgctaaacactgggcttcatcacccagggataaaaacagagatcattgtcttggacctcctgcatcagcctattcaaaattatctctctctctagctttccacaaatcctaaaattcctgtcccaagccacccaaattctcagatcttttctggaacaaggcagaatataaaataaatatacatttagtggcttgggctatggtctccaaagatccttcaaaaatacatcaagccagcttcattcactcactttacttagaacagagatataagggcctgggatgcatttattttatcaataccaatttttgtggccatggcagacattgctaatcaatcacagcactatttcctattaagcccactgatttcttcacaatccttctcaaattacaattccaaagagccgccactcaacagtcagatgaacccaacagtcagatgagagaaatgaaccctacttgctatctctatcttagaaagcaaaaacaaacaggagtttccagggagaatgggaaagccagggggcataaaaggtacagtcaggggaaaatagatctaggcagagtgccttagtcagggaccacgggcgctgaatctgcagtgccaacaccaaactgacacatctccaggtgtacctccaaccctagccttctcccacagctgcctacaacagagtctcccagccttctcagagagctaaaaccagaaatttccagactcatgaaagcaaccccccagcctctccccaaccctgccgcattgtctaatttttagaacactaggcttcttctttcatgtagttcctcataagcaggggccagaatatctcagccacctgcagtgacattgctggacccctgaaaaccattccataggagaatgggttccccaggctcacagtgtagagacattgagcccatcacaactgttttgactgctggcagtctaaaacagtccacccaccccatggcactgccgcgtgattcccgcggccattcagaagttcaagccgagatgctgacgttgctgagcaacgagatggtgagcatcagtgcaaatgcaccattcagcacatcagtcatatgcccagtgcagttacaagatgttgtttcggcaaagcattttgatggaatagggaactgcaaatgtatgatgattttgaaaaggctcagcaggatttgttcttaaaccgactcagtgtgtcatccccggttatttagaattacagttaagaaggagaaacttctataagactgtatgaacaaggtgatatcttcatagtgggctattacaggcaggaaaatgttttaactggtttacaaaatccatcaatacttgtgtcattccctgtaaaaggcaggagacatgtgattatgatcaggaaactgcacaaaattattgttttcagcccccgtgttattgtccttttgaactgtttttttttttattaaagccaaatttgtgttgtatatattcgtattccatgtgttagatggaagcatttcctatccagtgtgaataaaaagaacagttgtagtaaattattataaagccgatgatatttcatggcaggttattctaccaagctgtgcttgttggtttttcccatgactgtattgcttttataaatgtacaaatagttactgaaatgacgagacccttgtttgcacagcattaataagaaccttgataagaaccatattctgttgacagccagctcacagtttcttgcctgaagcttggtgcaccctccagtgagacacaagatctctcttttaccaaagttgagaacagagctggtggattaattaatagtcttcgatatctggccatgggtaacctcattgtaactatcatcagaatgggcagagatgatcttgaagtgtcacatacactaaagtccaaacactatgtcagatgggggtaaaatccattaaagaacaggaaaaaataattataagatgataagcaaatgtttcagcccaatgtcaacccagttaaaaaaaaaattaatgctgtgtaaaatggttgaattagtttgcaaactatataaagacatatgcagtaaaaagtctgttaatgcacatcctgtgggaatggagtgttctaaccaattgccttttcttgttatctgagctctcctatattatcatactcagataaccaaattaaaagaattagaatatgatttttaatacacttaacattaaactcttctaactttcttctttctgtgataattcagaagatagttatggatcttcaatgcctctgagtcattgttataaaaaatcagttatcactataccatgctataggagactgggcaaaacctgtacaatgacaaccctggaagttgctttttttaaaaaaataataaatttcttaaatcaactcttttttctggttgtctgtttgttataaagtgcaacgtattcaagtcctcaatatcctgatcataataccatgctataggagactgggcaaaacctgtacaatgacaaccctggaagttgcttttttaaaaaaaataataaatttcttaaatcaaaaaaaaaaaaaaaaaa

SEQ ID NO: 2 - protein, 564 aa, Homo sapiens
MATAERRALGIGFQWLSLATLVLICAGQGGRREDGGPACYGGFDLYFILDKSGSVLHHWNEIYYFVEQLAHKFISPQLRMSFIVFSTRGTTLMKLTEDREQIRQGLEELQKVLPGGDTYMHEGFERASEQIYYENRQGYRTASVIIALTDGELHEDLFFYSEREANRSRDLGAIVYCVGVKDFNETQLARIADSKDHVFPVNDGFQALQGIIHSILKKSCIEILAAEPSTICAGESFQVVVRGNGFRHARNVDRVLCSFKINDSVTLNEKPFSVEDTYLLCPAPILKEVGMKAALQVSMNDGLSFISSSVIITTTHCSDGSILAIALLILFLLLALALLWWFWPLCCTVIIKEVPPPPAEESEEEDDDGLPKKKWPTVDASYYGGRGVGGIKRMEVRWGEKGSTEEGAKLEKAKNARVKMPEQEYEFPEPRNLNNNMRRPSSPRKWYSPIKGKLDALWVLLRKGYDRVSVMRPQPGDTGRCINFTRVKNNQPAKYPLNNAYHTSSPPPAPIYTPPPPAPHCPPPPPSAPTPPIPSPPSTLPPPPQAPPPNRAPPPSRPPPRPSV

SEQ ID NO: 3 - DNA, 1667 bp, Homo sapiens
atcatatttaaaatctgggacaaagaaccgtcgggacggaactccttccattgcaaaagctcggcgcggcctcgggagctgcccggcggccccggaccgaggcagccctcccctttaaaagaagcggaggacaggattgggatccttgaaacccgaaacccagaaacagcatcggagcggaaaccagaggggaaaccttgaactcctccagacaattgcttccggggagttgcgagggagcgagggggaataaaggacccgcgaggaagggcccgcggatggcgcgtccctgagggtcgtggcgagttcgcggagcgtgggaaggagcggaccctgctctccccgggctgcgggccatggccacggcggagcggagagccctcggcatcggcttccagtggctctctttggccactctggtgctcatctgcgccgggcaagggggacgcagggaggatgggggtccagcctgctacggcggatttgacctgtacttcattttggacaaatcaggaagtgtgctgcaccactggaatgaaatctattactttgtggaacagttggctcacaaattcatcagcccacagttgagaatgtcctttattgttttctccacccgaggaacaaccttaatgaaactgacagaagacagagaacaaatccgtcaaggcctagaagaactccagaaagttctgccaggaggagacacttacatgcatgaaggatttgaaagggccagtgagcagatttattatgaaaacagacaagggtacaggacagccagcgtcatcattgctttgactgatggagaactccatgaagatctctttttctattcagagagggaggctaataggtctcgagatcttggtgcaattgtttactgtgttggtgtgaaagatttcaatgagacacagctggcccggattgcggacagtaaggatcatgtgtttcccgtgaatgacggctttcaggctctgcaaggcatcatccactcaattttgaagaagtcctgcatcgaaattctagcagctgaaccatccaccatatgtgcaggagagtcatttcaagttgtcgtgagaggaaacggcttccgacatgcccgcaacgtggacagggtcctctgcagcttcaagatcaatgactcggtcacactcaatgagaagcccttttctgtggaagatacttatttactgtgtccagcgcctatcttaaaagaagttggcatgaaagctgcactccaggtcagcatgaacgatggcctctcttttatctccagttctgtcatcatcaccaccacacactgttctgacggttccatcctggccatcgccctgctgatcctgttcctgctcctagccctggctctcctctggtggttctggcccctctgctgcactgtgattatcaaggaggtccctccaccccctgccgaggagagtgaggaaaataaaataaaataacaagaagaagaaagaaagaaatcccacagaaacagataacctaacacagcccgtgcaacgtattttatacaatgctctgaaaatcatagtctcaatctagacagtcttttcctctagttccctgtattcaaatcccagtgtctaacattcaataaatagctatatgaaatcaaaaaaaaaaaaaaaaaaaaaaaaaaaaaaaaa

SEQ ID NO: 4 - protein, 368 aa, Homo sapiens
MATAERRALGIGFQWLSLATLVLICAGQGGRREDGGPACYGGFDLYFILDKSGSVLHHWNEIYYFVEQLAHKFISPQLRMSFIVFSTRGTTLMKLTEDREQIRQGLEELQKVLPGGDTYMHEGFERASEQIYYENRQGYRTASVIIALTDGELHEDLFFYSEREANRSRDLGAIVYCVGVKDFNETQLARIADSKDHVFPVNDGFQALQGIIHSILKKSCIEILAAEPSTICAGESFQVVVRGNGFRHARNVDRVLCSFKINDSVTLNEKPFSVEDTYLLCPAPILKEVGMKAALQVSMNDGLSFISSSVIITTTHCSDGSILAIALLILFLLLALALLWWFWPLCCTVIIKEVPPPPAEESEENKIK

SEQ ID NO: 5 - DNA, 2360 bp, Homo sapiens
atcatatttaaaatctgggacaaagaaccgtcgggacggaactccttccattgcaaaagctcggcgcggcctcgggagctgcccggcggccccggaccgaggcagccctcccctttaaaagaagcggaggacaggattgggatccttgaaacccgaaacccagaaacagcatcggagcggaaaccagaggggaaaccttgaactcctccagacaattgcttccggggagttgcgagggagcgagggggaataaaggacccgcgaggaagggcccgcggatggcgcgtccctgagggtcgtggcgagttcgcggagcgtgggaaggagcggaccctgctctccccgggctgcgggccatggccacggcggagcggagagccctcggcatcggcttccagtggctctctttggccactctggtgctcatctgcgccgggcaagggggacgcagggaggatgggggtccagcctgctacggcggatttgacctgtacttcattttggacaaatcaggaagtgtgctgcaccactggaatgaaatctattactttgtggaacagttggctcacaaattcatcagcccacagttgagaatgtcctttattgttttctccacccgaggaacaaccttaatgaaactgacagaagacagagaacaaatccgtcaaggcctagaagaactccagaaagttctgccaggaggagacacttacatgcatgaaggatttgaaagggccagtgagcagatttattatgaaaacagacaagggtacaggacagccagcgtcatcattgctttgactgatggagaactccatgaagatctctttttctattcagagagggaggctaataggtctcgagatcttggtgcaattgtttactgtgttggtgtgaaagatttcaatgagacacagctggcccggattgcggacagtaaggatcatgtgtttcccgtgaatgacggctttcaggctctgcaaggcatcatccactcaattttgaagaagtcctgcatcgaaattctagcagctgaaccatccaccatatgtgcaggagagtcatttcaagttgtcgtgagaggaaacggcttccgacatgcccgcaacgtggacagggtcctctgcagcttcaagatcaatgactcggtcacactcaatgagaagcccttttctgtggaagatacttatttactgtgtccagcgcctatcttaaaagaagttggcatgaaagctgcactccaggtcagcatgaacgatggcctctcttttatctccagttctgtcatcatcaccaccacacactgtagcctccacaaaattgcatcaggccccacaacagctgcttgcatggaatagcagagaataccgcctgctccctccggacagcacactcctgaaaacggggagagaggagccaaacatgctcggtttacactttccttatttactgaatgagtggagggcagagacaggcctggagttacgcacactgagtgccccaacatggaaagaaacatcaggagggacaggaaacgttccctccttaaccaacagttttcaagaccttactggaggcactttattggctacataatcactccatgcggtgggcatcaggcagaatcctggtgcagacccaactttgaggtggaggatttcacagtttctttattttgaacttcccccaggctcccactaattcctctccattctatcctcctccctttcccacaaaagaaaacagaaaggagcagcagtgtttgataccgtatcatccagaggcctggttctctcccattatagggcaaacaagccctggcaagatatttcactcccgccccatgccatgcattaaaaatccaaaattgcctatattccacctgccaagcaagagatgctttcattattgaagttccaaatgtatacctttgagaacagtgccttctcgtcttaaaagagaggtcctcattttgtgagttgggagcagagggaattaaagaaagccatgatgcagggatttggccattcaagccgggcagccttcagagaatgtcatccctaatgacacatgcccgaatgaaggagcggggctgagcttgtcctgccttcgtattgaatgttgcctgtctgcctccttaatagcgggcctctgtgtgagcatttgacaagacttaaaactattcattgaagaaaatggatgatcccccaacaggaagatgcaaccccatgggctgcctgcttgaccacagaagtgcttccagctccagttgctcatctgagaactccccccaccacttgctgttaaaattgttaaaattaaaggccatgttgattgattttttaaaaaaaaaaaaaaaa

SEQ ID NO: 6 - protein, 333 aa, Homo sapiens
MATAERRALGIGFQWLSLATLVLICAGQGGRREDGGPACYGGFDLYFILDKSGSVLHHWNEIYYFVEQLAHKFISPQLRMSFIVFSTRGTTLMKLTEDREQIRQGLEELQKVLPGGDTYMHEGFERASEQIYYENRQGYRTASVIIALTDGELHEDLFFYSEREANRSRDLGAIVYCVGVKDFNETQLARIADSKDHVFPVNDGFQALQGIIHSILKKSCIEILAAEPSTICAGESFQVVVRGNGFRHARNVDRVLCSFKINDSVTLNEKPFSVEDTYLLCPAPILKEVGMKAALQVSMNDGLSFISSSVIITTTHCSLHKIASGPTTAACME